SEQUENCE LISTING (38 sequences)

SEQ ID NO: 1 - protein, 255 aa, Homo sapiens [sequence omitted]

SEQ ID NO: 2 - protein, 3 aa, Artificial Sequence
  Synthetic Construct
EPA

SEQ ID NO: 3 - protein, 4 aa, Artificial Sequence
  Synthetic Construct
VPPE

SEQ ID NO: 4 - protein, 3 aa, Artificial Sequence
  Synthetic Construct
EPH

SEQ ID NO: 5 - protein, 3 aa, Artificial Sequence
  Synthetic Construct
EPE

SEQ ID NO: 6 - protein, 3 aa, Artificial Sequence
  Synthetic Construct
VPV

SEQ ID NO: 7 - protein, 3 aa, Artificial Sequence
  Synthetic Construct
EPQ

SEQ ID NO: 8 - protein, 3 aa, Artificial Sequence
  Synthetic Construct
GPT

SEQ ID NO: 9 - protein, 3 aa, Artificial Sequence
  Synthetic Construct
NPD

SEQ ID NO: 10 - protein, 3 aa, Artificial Sequence
  Synthetic Construct
EPV

SEQ ID NO: 11 - protein, 3 aa, Artificial Sequence
  Synthetic Construct
EPL

SEQ ID NO: 12 - protein, 4 aa, Artificial Sequence
  Synthetic Construct
APPE

SEQ ID NO: 13 - protein, 3 aa, Artificial Sequence
  Synthetic Construct
NPV

SEQ ID NO: 14 - protein, 3 aa, Artificial Sequence
  Synthetic Construct
FPV

SEQ ID NO: 15 - protein, 4 aa, Artificial Sequence
  Synthetic Construct
VPPD

SEQ ID NO: 16 - protein, 7 aa, Artificial Sequence
  Synthetic Construct
EPAVPPE

SEQ ID NO: 17 - protein, 9 aa, Artificial Sequence
  Synthetic Construct
VPPEEAEPH

SEQ ID NO: 18 - protein, 7 aa, Artificial Sequence
  Synthetic Construct
EPHTEPE

SEQ ID NO: 19 - protein, 8 aa, Artificial Sequence
  Synthetic Construct
EPEEQVPV

SEQ ID NO: 20 - protein, 8 aa, Artificial Sequence
  Synthetic Construct
VPVEAEPQ

SEQ ID NO: 21 - protein, 7 aa, Artificial Sequence
  Synthetic Construct
GPTGEPQ

SEQ ID NO: 22 - protein, 8 aa, Artificial Sequence
  Synthetic Construct
NPDSSEPV

SEQ ID NO: 23 - protein, 8 aa, Artificial Sequence
  Synthetic Construct
APPEDNPV

SEQ ID NO: 24 - protein, 12 aa, Artificial Sequence
  Synthetic Construct
FPVEEQQEVPPD

SEQ ID NO: 25 - protein, 10 aa, Artificial Sequence
  Synthetic Construct
DGPTGEPQQE

SEQ ID NO: 26 - protein, 11 aa, Artificial Sequence
  Synthetic Construct
EQENPDSSEPV

SEQ ID NO: 27 - protein, 10 aa, Artificial Sequence
  Synthetic Construct
APPEDNPVED

SEQ ID NO: 28 - protein, 9 aa, Artificial Sequence
  Synthetic Construct
EEQQEVPPD

SEQ ID NO: 29 - protein, 22 aa, Artificial Sequence
  Synthetic Construct
GEDLQQEDGPTGEPQQEDDEFL

SEQ ID NO: 30 - protein, 22 aa, Artificial Sequence
  Synthetic Construct
DMEEMMSEQENPDSSEPVVEDE

SEQ ID NO: 31 - protein, 22 aa, Artificial Sequence
  Synthetic Construct
NEGIEITEVTAPPEDNPVEDSQ

SEQ ID NO: 32 - protein, 23 aa, Artificial Sequence
  Synthetic Construct
DSQVIVEEVSIFPVEEQQEVPPD

SEQ ID NO: 33 - protein, 16 aa, Artificial Sequence
  Synthetic Construct
KSSQSLVYSDGKTYLN

SEQ ID NO: 34 - protein, 7 aa, Artificial Sequence
  Synthetic Construct
LVSKLDS

SEQ ID NO: 35 - protein, 9 aa, Artificial Sequence
  Synthetic Construct
LQGSHFPRT

SEQ ID NO: 36 - protein, 10 aa, Artificial Sequence
  Synthetic Construct
GFTFSRYTMS

SEQ ID NO: 37 - protein, 17 aa, Artificial Sequence
  Synthetic Construct
YISNGGRSIYYADSVKG

SEQ ID NO: 38 - protein, 4 aa, Artificial Sequence
  Synthetic Construct
AMDN

(19) United States
(12) Patent Application Publication
(10) Pub. No.: US 2022/0089719 A1
(43) Pub. Date: Mar. 24, 2022

(54) CONSTRUCTS TARGETING LABYRINTHIN OR A PORTION THEREOF AND USES THEREOF

(71) Applicant: LabyRx Immunologic Therapeutics (USA) Limited, Aubum, CA (US)

(72) Inventors: Michael BABICH, Auburn, CA (US); James A. RADOSEVICH, Belvidere, IL (US)

(73) Assignee: LabyRx Immunologic Therapeutics (USA) Limited, Aubum, CA (US)

(21) Appl. No.: 17/309,955

(22) PCT Filed: Jan. 7, 2020
(86) PCT No.: PCT/US2020/012613

§ 371 Date: Jul. 2, 2021

Related U.S. Application Data
(60) Provisional application No. 62/789,871, filed on Jan. 8, 2019.

(51) Int. Cl. C07K 16/28; A61K 35/17; G01N 33/574

(52) U.S. Cl. CPC C07K 16/28; A61K 35/17; C07K 2317/92; G01N 33/57423; C07K 2317/622; G01N 33/57492

(57) ABSTRACT

Constructs comprising an antibody moiety that specifically binds to Labyrinthin or a portion thereof and an effector domain are provided. Also provided are methods of making and using these constructs and compositions thereof.

### CROSS-REFERENCE TO RELATED APPLICATIONS

This application claims priority to U.S. Provisional Patent Application No. 62/789,871, filed on Jan. 8, 2019, the disclosure of which is hereby incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 185722000240SEQLIST.TXT, date recorded: Jan. 7, 2020, size: 9 KB).

### TECHNICAL FIELD

The present application relates to constructs comprising an antibody moiety that specifically binds to Labyrinthin or a portion thereof and an effector domain. Also provided are methods of making and using these constructs and compositions thereof.

### BACKGROUND

Historically, cancer has been characterized largely based on the tissue type or organ in which the cancer originates, e.g., lung cancer, breast cancer, and colon cancer. Many cancer treatments are also based on the tissue or organ-based classification of the cancer. It is well recognized that such tissue or organ-based classification of cancers may not provide sufficient guidance for selection of an efficacious treatment. This is due, in part, to the finding that cancers originating from a single tissue type or organ may be highly heterogeneous, and such differences may require a personalized cancer treatment approach. For example, defining cancers by a biomarker, as opposed to a tissue type or organ of origin, may allow for improved cancer treatment, e.g., triple negative breast cancer, which lacks expression of estrogen receptors, progesterone receptors, and HER2/neu, is not responsive to traditional hormone-based therapies that target any one or more of the identified receptors and requires alternative treatments. After identification of a cancer subtype based on a biomarker, significant research is required to develop novel agents for efficacious treatment of such a cancer subtype.

One such identified cancer subtype is a Labyrinthin-expressing cancer. Labyrinthin is a cell surface protein expressed on the extracellular surface of the plasma membrane of some cancers, such as adenocarcinomas. Cell surface expression of Labyrinthin is not cell cycle specific. Furthermore, Labyrinthin is not found in the serum of normal or tumor bearing patients, and is not shed into the culture media by Labyrinthin positive cell lines.

Immunotherapy in humans has been limited, in part due to adverse responses to non-human monoclonal antibodies. Early clinical trials using rodent antibodies revealed human anti-mouse antibody (HAMA) and human anti-rat antibody (HARA) responses, which lead to rapid clearance of the antibody. Less immunogenic antibodies have since been developed, including chimeric antibodies, humanized antibodies, PRIMATIZED® antibodies, and human antibodies prepared using transgenic mice or phage display libraries. See, e.g., Morrison et al., Proc Natl Acad Sci USA, 81, 1984; and Queen et al. Proc Natl Acad Sci USA, 86, 1989. Avoidance of a HAMA response permits high dose and repeated dose administration to achieve a therapeutic response.

Recent advances in using phage display to generate mAbs have made it possible to select agents with exquisite specificity against defined epitopes from large antibody repertoires. A number of such mAbs specific for solid tumor antigens, in the context of HLA-A01 and HLA-A02, have been successfully selected from phage display libraries (Noy et al., Expert Rev Anticancer Ther, 5, 2005: Chames et al., Proc Natl Acad Sci USA, 97, 2000; Held et al., Eur J Immunol, 34, 2004; Lev et al., Cancer Res, 62, 2002; Klechevsky et al., Cancer Res, 68, 2008). More recently, a human mAb specific for human WT1/HLA-A02 complex, a well-described T cell epitope, has been shown to inhibit multiple cancer cell lines and primary cancer cells via Fc-mediated effector cell function (Dao et al., Sci Transl Med, 5, 2013; Veomett et al, Clin Cancer Res, 2014) in cellular assays and in in vivo models.

All references cited herein, including patent applications and publications, are incorporated by reference in their entirety.

### BRIEF SUMMARY

In one aspect, the present application provides isolated anti-Labyrinthin constructs comprising an antibody moiety that specifically binds to Labyrinthin or a portion thereof and an effector domain.

In some embodiments, the antibody moiety is conjugated to the effector domain. In some embodiments, the effector domain comprises an effector molecule. In some embodiments, the effector molecule comprises a therapeutic agent. In some embodiments, the therapeutic agent is selected from the group consisting of a drug, a toxin, a radioisotope, a protein, a peptide, and a nucleic acid. In some embodiments, the isolated anti-Labyrinthin construct is an antibody drug conjugate (ADC).

In some embodiments, the effector domain comprises a diagnostic agent. In some embodiments, the diagnostic agent is a label.

In some embodiments, the antibody moiety is fused to an effector domain.

In some embodiments, the isolated anti-Labyrinthin construct is a chimeric antigen receptor (CAR) comprising an extracellular domain comprising the antibody moiety fused to the effector domain, wherein the effector domain comprises a transmembrane domain and an intracellular signaling domain.

In some embodiments, the isolated anti-Labyrinthin construct is a bispecific T-cell engager (BiTE) comprising an extracellular domain comprising the antibody moiety fused to an anti-CD3 antibody or fragment thereof.

In some embodiments, the isolated anti-Labyrinthin construct is a T-cell receptor (TCR) comprising an extracellular domain comprising the antibody moiety fused to the effector domain, wherein the effector domain comprises a transmembrane domain and/or an intracellular signaling domain of a TCR subunit.

In some embodiments, the antibody moiety is a full-length antibody, a Fab, a Fab′, a (Fab′)2, an Fv, or a single chain Fv (scFv). In some embodiments, the antibody moiety binds Labyrinthin or a portion thereof with a Kd from about 0.1 pM to about 500 nM.

In some embodiments, the antibody moiety specifically binds to a Labyrinthin-derived peptide comprising an amino acid sequence selected from the group consisting of: SEQ ID NOs:2-32. In some embodiments, the Labyrinthin-derived peptide comprises a B-cell and T-cell epitope. In some embodiments, the Labyrinthin-derived peptide comprises a B-cell epitope. In some embodiments, the Labyrinthin-derived peptide comprises a T-cell epitope.

In some embodiments, the antibody moiety is multispecific. In some embodiments, the multispecific antibody moiety is a tandem scFv, a diabody (db), a single chain diabody (scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody. In some embodiments, the multispecific antibody moiety is a tandem scFv comprising two scFvs linked by an optional peptide linker.

In another aspect, the present application provides pharmaceutical compositions comprising any of the isolated anti-Labyrinthin constructs described herein.

In another aspect, the present application provides host cells expressing any of the isolated anti-Labyrinthin constructs described herein.

In another aspect, the present application provides nucleic acids encoding the polypeptide components of any of the isolated anti-Labyrinthin constructs described herein.

In another aspect, the present application provides effector cells comprising any of the nucleic acids described herein. In some embodiments, the effector cell is a T cell.

In another aspect, the present application provides methods of detecting a cell presenting Labyrinthin or a portion thereof on its surface, comprising: (a) contacting the cell with an isolated anti-Labyrinthin construct, wherein the isolated anti-Labyrinthin construct comprises an effector domain comprising a label; and (b) detecting the presence of the label on the cell.

In another aspect, the present application provides methods of treating an individual having a Labyrinthin-positive disease, comprising administering to the individual: (a) an effective amount of any of the pharmaceutical compositions described herein; or (b) an effective amount of any of the effector cells described herein. In some embodiments, the Labyrinthin status is used as a basis for selecting the individual for treatment.

In another aspect, the present application provides methods of diagnosing an individual having a Labyrinthin-positive disease, comprising: (a) administering an effective amount of an isolated anti-Labyrinthin construct to the individual, wherein the isolated anti-Labyrinthin construct comprises an effector domain comprising a label: and (b) determining the level of the label in the individual, wherein a level of the label above a threshold level indicates that the individual has the Labyrinthin-positive disease.

In another aspect, the present application provides methods of diagnosing an individual having a Labyrinthin-positive disease, comprising: (a) contacting a sample derived from the individual with any of the isolated anti-Labyrinthin constructs described herein; and (b) identifying one or more cells bound with the isolated anti-Labyrinthin construct in the sample, thereby diagnosing an individual having a Labyrinthin-positive disease.

In some embodiments, the Labyrinthin-positive disease is Labyrinthin-positive cancer. In some embodiments, the Labyrinthin-positive cancer is an adenocarcinoma.

### BRIEF DESCRIPTION OF THE DRAWINGS

FIGS. 1A-1B show time versus binding response measurements of the MCA 44-3A6 mouse monoclonal IgG antibody (FIG. 1A) and the X373 scFV derivative (FIG. 1B).

FIG. 2 shows a western immunoblot of lysates from A549 lung adenocarcinoma cells (positive control) and WI38 fibroblasts (negative control) using X509Fab.

### DETAILED DESCRIPTION

The present application provides, in some aspects, isolated constructs (referred to as “anti-Labyrinthin constructs” or “anti-LAB constructs”) that comprise an antibody moiety that specifically binds to Labyrinthin or a portion thereof and an effector domain. Also provided are methods of making and using these constructs and compositions thereof.

The present application is based, in part, on the finding that specific cancers, such as adenocarcinomas, selectively express cell surface Labyrinthin and that Labyrinthin-positive cancers do not shed Labyrinthin. Furthermore, normal, non-cancerous cancers do not express cell surface Labyrinthin. Therefore, Labyrinthin represents a useful marker for defining Labyrinthin-positive cancers and target for immunotherapy based treatments.

Thus, in some embodiments, the present application provides isolated anti-Labyrinthin constructs comprising an antibody moiety that specifically binds to Labyrinthin or a portion thereof and an effector domain.

In some embodiments, the isolated anti-Labyrinthin construct is a chimeric antigen receptor (CAR) comprising an extracellular domain comprising the antibody moiety fused to the effector domain, wherein the effector domain comprises a transmembrane domain and an intracellular signaling domain.

In some embodiments, the isolated anti-Labyrinthin construct is a bispecific T-cell engager (BiTE) comprising an extracellular domain comprising the antibody moiety fused to an anti-CD3 antibody or fragment thereof.

In some embodiments, the isolated anti-Labyrinthin construct is a T-cell receptor (TCR) comprising an extracellular domain comprising the antibody moiety fused to the effector domain, wherein the effector domain comprises a transmembrane domain and/or an intracellular signaling domain of a TCR subunit.

In some embodiments, the present application provides pharmaceutical compositions comprising any of the isolated anti-Labyrinthin constructs described herein.

In some embodiments, the present application provides host cells expressing any of the isolated anti-Labyrinthin constructs described herein.

In some embodiments, the present application provides nucleic acids encoding the polypeptide components of any of the isolated anti-Labyrinthin constructs described herein.

In some embodiments, the present application provides effector cells comprising any of the nucleic acids described herein. In some embodiments, the effector cell is a T cell.

In some embodiments, the present application provides methods of detecting a cell presenting Labyrinthin or a portion thereof on its surface, comprising: (a) contacting the cell with an isolated anti-Labyrinthin construct, wherein the isolated anti-Labyrinthin construct comprises an effector domain comprising a label: and (b) detecting the presence of the label on the cell.

In some embodiments, the present application provides methods of treating an individual having a Labyrinthin-positive disease, comprising administering to the individual: (a) an effective amount of any of the pharmaceutical compositions described herein: or (b) an effective amount of any of the effector cells described herein.

In some embodiments, the present application provides methods of diagnosing an individual having a Labyrinthin-positive disease, comprising: (a) administering an effective amount of an isolated anti-Labyrinthin construct to the individual, wherein the isolated anti-Labyrinthin construct comprises an effector domain comprising a label; and (b) determining the level of the label in the individual, wherein a level of the label above a threshold level indicates that the individual has the Labyrinthin-positive disease.

In some embodiments, the present application provides methods of diagnosing an individual having a Labyrinthin-positive disease, comprising: (a) contacting a sample derived from the individual with any of the isolated anti-Labyrinthin constructs described herein; and (b) identifying one or more cells bound with the isolated anti-Labyrinthin construct in the sample, thereby diagnosing an individual having a Labyrinthin-positive disease.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### Definitions

The term “antibody moiety” includes full-length antibodies and antigen-binding fragments thereof. A full-length antibody comprises two heavy chains and two light chains. The variable regions of the light and heavy chains are responsible for antigen binding. The variable region in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody moiety “competes” for binding to a target Labyrinthin with a second antibody moiety when the first antibody moiety inhibits target Labyrinthin binding of the second antibody moiety by at least about 50% (such as at least about any of 55%, 60%, 65%, 70%, 75%, 80%, 85%, 90%, 95%, 98% or 99%) in the presence of an equimolar concentration of the first antibody moiety, or vice versa. A high throughput process for “binning” antibodies based upon their cross-competition is described in PCT Publication No. WO 03/48731.

As use herein, the term “specifically binds” or “is specific for” refers to measurable and reproducible interactions, such as binding between a target and an antibody or antibody moiety that is determinative of the presence of the target in the presence of a heterogeneous population of molecules, including biological molecules. For example, an antibody or antibody moiety that specifically binds to a target (which can be an epitope) is an antibody or antibody moiety that binds this target with greater affinity, avidity, more readily, and/or with greater duration than its bindings to other targets. In some embodiments, an antibody or antibody moiety that specifically binds to an antigen reacts with one or more antigenic determinants of the antigen (for example an Labyrinthin or a portion thereof) with a binding affinity that is at least about 10 times its binding affinity for other targets.

An “isolated” anti-Labyrinthin construct as used herein refers to an anti-Labyrinthin construct that (1) is not associated with proteins found in nature, (2) is free of other proteins from the same source, (3) is expressed by a cell from a different species, or, (4) does not occur in nature.

The term “isolated nucleic acid” as used herein is intended to mean a nucleic acid of genomic, cDNA, or synthetic origin or some combination thereof, which by virtue of its origin the “isolated nucleic acid” (1) is not associated with all or a portion of a polynucleotide in which the “isolated nucleic acid” is found in nature, (2) is operably linked to a polynucleotide which it is not linked to in nature, or (3) does not occur in nature as part of a larger sequence.

As used herein, the term “CDR” or “complementarity determining region” is intended to mean the non-contiguous antigen combining sites found within the variable region of both heavy and light chain polypeptides. These particular regions have been described by Kabat et al, J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

The term “chimeric antibodies” refer to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this invention (see U.S. Pat. No. 4,816,567; and Morrison et al., Proc. Natl. Acad. Sci. LISA, 81:6851-6855 (1984)).

The term “semi-synthetic” in reference to an antibody or antibody moiety means that the antibody or antibody moiety has one or more naturally occurring sequences and one or more non-naturally occurring (i.e., synthetic) sequences.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv.” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment. i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404.097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The terms “Fc receptor” or “FcR” are used to describe a receptor that binds to the Fc region of an antibody. In some embodiments, an FcR of this invention is one that binds an IgG antibody (a γ receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron. Annu. Rev. Immunol. 15:203-234 (1997)). The term includes allotypes, such as FcγRIIIA allotypes: FcγRIIIA-Phe158, FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol 24:249 (1994)).

The term “FcRn” refers to the neonatal Fc receptor (FcRn). FcRn is structurally similar to major histocompatibility complex (MIHC) and consists of an α-chain noncovalently bound to β2-microglobulin. The multiple functions of the neonatal Fc receptor FcRn are reviewed in Ghetie and Ward (2000) Annu. Rev. Immunol. 18, 739-766. FcRn plays a role in the passive delivery of immunoglobulin IgGs from mother to young and the regulation of serum IgG levels. FcRn can act as a salvage receptor, binding and transporting pinocytosed IgGs in intact form both within and across cells, and rescuing them from a default degradative pathway.

The “CH1 domain” of a human IgG Fc region (also referred to as “C1” of “H1” domain) usually extends from about amino acid 118 to about amino acid 215 (EU numbering system).

“Hinge region” is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region (also referred to as “C2” of “H2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C2” or “H3” domain) comprises the stretch of residues C-terminal to a CH2 domain in an Fc region (i.e. from about amino acid residue 341 to the C-terminal end of an antibody sequence, typically at amino acid residue 446 or 447 of an IgG).

A “functional Fc fragment” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art.

An antibody with a variant IgG Fe with “altered” FcR binding affinity or ADCC activity is one which has either enhanced or diminished FcR binding activity (e.g., FcγR or FcRn) and/or ADCC activity compared to a parent polypeptide or to a polypeptide comprising a native sequence Fc region. The variant Fc which “exhibits increased binding” to an FcR binds at least one FcR with higher affinity (e.g., lower apparent K_{d} or IC₅₀ value) than the parent polypeptide or a native sequence IgG Fc. According to some embodiments, the improvement in binding compared to a parent polypeptide is about 3 fold, such as about any of 5, 10, 25, 50, 60, 100, 150, 200, or up to 500 fold, or about 25% to 1000% improvement in binding. The polypeptide variant which “exhibits decreased binding” to an FcR, binds at least one FcR with lower affinity (e.g., higher apparent K_{d} or higher IC₅₀ value) than a parent polypeptide. The decrease in binding compared to a parent polypeptide may be about 40% or more decrease in binding.

“Antibody-dependent cell-mediated cytotoxicity” or “ADCC” refers to a form of cytotoxicity in which secreted Ig bound to Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally. ADCC activity of the molecule of interest may be assessed in vivo, e.g., in an animal model such as that disclosed in Clynes et al. PNAS (USA) 95:652-656 (1998).

The polypeptide comprising a variant Fc region which “exhibits increased ADCC” or mediates antibody-dependent cell-mediated cytotoxicity (ADCC) in the presence of human effector cells more effectively than a polypeptide having wild type IgG Fc or a parent polypeptide is one which in vitro or in vivo is substantially more effective at mediating ADCC, when the amounts of polypeptide with variant Fc region and the polypeptide with wild type Fc region (or the parent polypeptide) in the assay are essentially the same. Generally, such variants will be identified using any in vitro ADCC assay known in the art, such as assays or methods for determining ADCC activity, e.g. in an animal model etc. In some embodiments, the variant is from about 5 fold to about 100 fold, e.g. from about 25 to about 50 fold, more effective at mediating ADCC than the wild type Fc (or parent polypeptide).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al, J. Immunol. Methods 202:163 (1996), may be performed. Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See, also. Idusogie et al. J. Immunol. 164: 4178-4184 (2000).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “substantially homologous,” as used herein, refers to the sequence similarity of a sequence disclosed herein as compared to a reference sequence, wherein the sequence has at least about 85% similarity (e.g., homology) with a reference or a portion thereof, such as at least about any of 86% similarity, 87% similarity, 88% similarity, 89% similarity, 90% similarity, 91% similarity, 92% similarity, 93% similarity, 94% similarity, 95% similarity, 96% similarity, 97% similarity, 98% similarity, 99% similarity, or 100% similarity. Methods for determining sequence similarity are known in the art, e.g., as described in Pearson, W. R., Curr Protoc Bioinformatics, 2013.

The term “label” when used herein refers to a detectable compound or composition which can be conjugated directly or indirectly to the anti-Labyrinthin antibody moiety. The label may be detectable by itself (e.g., radioisotope labels or fluorescent labels) or, in the case of an enzymatic label, may catalyze chemical alteration of a substrate compound or composition which is detectable.

As used herein, “treatment” or “treating” is an approach for obtaining beneficial or desired results, including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the invention contemplate any one or more of these aspects of treatment.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

“Activation”, as used herein in relation to T cells, refers to the state of a T cell that has been sufficiently stimulated to induce detectable cellular proliferation. Activation can also be associated with induced cytokine production, and detectable effector functions.

The term “effective amount,” as used herein, refers to an amount of a compound or composition sufficient to treat a specified disorder, condition, or disease, such as ameliorate, palliate, lessen, and/or delay one or more symptoms of the disorder, condition, or disease. In reference to cancer, an effective amount comprises an amount sufficient to, e.g., cause a tumor to shrink and/or to decrease the growth rate of the tumor (such as to suppress tumor growth) or to prevent or delay other unwanted cell proliferation in the cancer. In some embodiments, an effective amount is an amount sufficient to delay development of cancer. In some embodiments, an effective amount is an amount sufficient to prevent or delay recurrence. An effective amount can be administered in one or more administrations. In the case of cancer, the effective amount of the drug or composition may: (i) reduce the number of cancerous cells; (ii) reduce tumor size; (iii) inhibit, retard, slow to some extent and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, “delaying” the development of cancer means to defer, hinder, slow, retard, stabilize, and/or postpone development of the disease. This delay can be of varying lengths of time, depending on the history of the disease and/or individual being treated. As is evident to one of ordinary skill in the art, a sufficient or significant delay can, in effect, encompass prevention, in that the individual does not develop the disease. A method that “delays” development of cancer is a method that reduces the probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “individual” refers to a mammal and includes, but is not limited to, human, bovine, horse, feline, canine, rodent, or primate.

The term “based on” or “basis for,” as used herein, includes assessing, determining, obtaining, or measuring one or more characteristic of an individual or a cancer therein as described herein, and in some embodiments, selecting the individual suitable for receiving a treatment as described in the methods disclosed herein. For example, when a Labyrinthin status of a cancer is used as a basis for selecting an individual for a treatment method herein, assessing (or aiding in assessing), measuring, obtaining, or determining the Labyrinthin status may be included in a method of a treatment as described herein, e.g., the Labyrinthin status is measured before and/or during and/or after treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage: or (g) predicting likelihood of clinical benefits.

The basis or bases disclosed herein for use with the methods of the present application, such a Labyrinthin status, may, in some aspects, be based on a comparison to a control. In some embodiments, control is a known standard obtained from the literature (e.g., a known gene sequence, RNA sequence, protein sequence, gene expression level). In some embodiments, the control is a control sample obtained from the individual to be, or being, treated using the methods disclosed herein (e.g., a control sample from a non-cancerous tissue). In some embodiments, the control is a control sample obtained from an individual other than the individual to be, or being, treated using the methods disclosed herein (e.g., a control sample from a healthy volunteer or a volunteer not having cancer). In some embodiments, the control is obtained from a given patient population. For example, regarding a level of gene expression or enzyme activity level, a control level may be the median expression level of that gene or the median enzyme activity level of that enzyme for the patient population. And, for example, if the expression level of a gene of interest for the single patient is determined to be above the median expression level of the patient population, that patient is determined to have high expression of the gene of interest. Alternatively, if the expression level of a gene of interest for the single patient is determined to be below the median expression level of the patient population, that patient is determined to have low expression of the gene of interest. In some embodiments, the single patient has a disease (such as cancer) and the patient population does not have the disease. In some embodiments, the single patient and the patient population have the same histological type of a disease. A population may be about, or alternatively at least about any of the following, in terms of number of individuals measured: 2, 5, 10, 15, 20, 25, 30, 50, 60, 75, 100, 125, 150, 175, 200, 225, 250, 300, 400, 500. Preferably, a sufficient number of individuals are measured to provide a statistically significant population, which can be determined by methods known in the art. In some embodiments, the population is a group participating in a clinical trial.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### Anti-LAB Constructs

The present application provides anti-Labyrinthin constructs (referred to herein as “anti-LAB constructs”), such as isolated anti-LAB constructs, comprising an antibody moiety that specifically binds to Labyrinthin or a portion thereof and an effector domain. In some embodiments, the antibody moiety is conjugated to the effector domain. For example, the effector domain can comprise an effector molecule which can be a therapeutic agent or a label. In some embodiments, the therapeutic agent is selected from the group consisting of a drug, a toxin, a radioisotope, a protein, a peptide, and a nucleic acid. In some embodiments, the effector molecule is a diagnostic agent such as a label. Conjugation of effector molecules are well-known in the art, and can include covalent conjugation (e.g., via a chemical bond or via a linker) or non-covalent interactions (e.g., via biotin/streptavidin and other protein-protein interaction pairs).

In some embodiments, the anti-Labyrinthin construct is a fusion protein. For example, the anti-Labyrinthin antibody moiety can be part of a CAR (chimeric antigen receptor), an engineered TCR, or a bispecific T cell engager molecule. These various constructs are discussed in more details in the sections below.

### Anti-LAB CAR

The present application in one aspect provides a chimeric antigen receptor (CAR) comprising an antibody moiety that specifically binds to Labyrinthin or a portion thereof (herein referred to as “anti-LAB CAR”). Also provided are CAR effector cells (e.g., T cells) comprising a CAR comprising an anti-LAB antibody moiety (also referred to herein as an “anti-LAB CAR effector cell”, e.g., “anti-LAB CAR T cell”).

The anti-LAB CAR comprises (a) an extracellular domain comprising an anti-LAB antibody moiety that specifically binds to Labyrinthin and (b) an intracellular signaling domain. A transmembrane domain may be present between the extracellular domain and the intracellular domain. Between the extracellular domain and the transmembrane domain of the anti-LAB CAR, or between the intracellular domain and the transmembrane domain of the anti-LAB CAR, there may be a linker or a spacer domain. The linker or spacer domain can be any oligo- or polypeptide that functions to link the transmembrane domain to the extracellular domain or the intracellular domain in the polypeptide chain. A spacer domain may comprise up to about 300 amino acids, including for example about 10 to about 100, or about 25 to about 50 amino acids.

The transmembrane domain may be derived either from a natural or from a synthetic source. Where the source is natural, the domain may be derived from any membrane-bound or transmembrane protein. Transmembrane regions of particular use in this invention may be derived from (i.e., comprise at least the transmembrane region(s) of) the α, β, δ, or γ chain of the T-cell receptor, CD28, CD3ε, CD3ζ, CD45, CD4, CD5, CD8. CD9, CD16. CD22, CD33, CD37, CD64, CD80, CD86, CD134, CD137, or CD154. In some embodiments, the transmembrane domain may be synthetic, in which case it may comprise predominantly hydrophobic residues such as leucine and valine. In some embodiments, a triplet of phenylalanine, tryptophan and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the intracellular signaling domain of the anti-LAB CAR. In some embodiments, the linker is a glycine-serine doublet.

In some embodiments, the transmembrane domain that naturally is associated with one of the sequences in the intracellular domain of the anti-LAB CAR is used (e.g., if an anti-LAB CAR intracellular domain comprises a CD28 co-stimulatory sequence, the transmembrane domain of the anti-LAB CAR is derived from the CD28 transmembrane domain). In some embodiments, the transmembrane domain can be selected or modified by amino acid substitution to avoid binding of such domains to the transmembrane domains of the same or different surface membrane proteins to minimize interactions with other members of the receptor complex.

The intracellular signaling domain of the anti-LAB CAR is responsible for activation of at least one of the normal effector functions of the immune cell in which the anti-LAB CAR has been placed in. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use in the anti-LAB CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that in some embodiments signals generated through the TCR alone are insufficient for full activation of the T cell and that a secondary or co-stimulatory signal may also be required. Thus, T cell activation can in some embodiments may be mediated by two distinct classes of intracellular signaling sequence: those that initiate antigen-dependent primary activation through the TCR (primary signaling sequences) and those that act in an antigen-independent manner to provide a secondary or co-stimulatory signal (co-stimulatory signaling sequences). Primary signaling sequences regulate primary activation of the TCR complex either in a stimulatory way, or in an inhibitory way. Primary signaling sequences that act in a stimulatory manner may contain signaling motifs which are known as immunoreceptor tyrosine-based activation motifs or ITAMs. The anti-LAB CAR constructs in some embodiments comprise one or more ITAMs. Examples of ITAM containing primary signaling sequences that are of particular use in the invention include those derived from TCRζ, FcRγ, FcRβ, CD3γ, CD3δ, CD3ε, CD5, CD22, CD79a, CD79b, and CD66d.

In some embodiments, the anti-LAB CAR comprises a primary signaling sequence derived from CD3ζ. For example, the intracellular signaling domain of the CAR can comprise the CD3ζ intracellular signaling sequence by itself or combined with any other desired intracellular signaling sequence(s) useful in the context of the anti-LAB CAR of the invention. For example, the intracellular domain of the anti-LAB CAR can comprise a CD3ζ intracellular signaling sequence and a costimulatory signaling sequence. The costimulatory signaling sequence can be a portion of the intracellular domain of a costimulatory molecule including, for example, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, and the like.

In some embodiments, the intracellular signaling domain of the anti-LAB CAR comprises the intracellular signaling sequence of CD3ζ and the intracellular signaling sequence of CD28. In some embodiments, the intracellular signaling domain of the anti-LAB CAR comprises the intracellular signaling sequence of CD3ζ and the intracellular signaling sequence of 4-1BB. In some embodiments, the intracellular signaling domain of the anti-LAB CAR comprises the intracellular signaling sequence of CD3ζ and the intracellular signaling sequences of CD28 and 4-1BB.

Thus, for example, in some embodiments, there is provided an anti-LAB CAR comprising: (a) an extracellular domain comprising an anti-LAB antibody moiety that specifically binds to Labyrinthin, (b) a transmembrane domain, and (c) an intracellular signaling domain. In some embodiments, the intracellular signaling domain is capable of activating an immune cell. In some embodiments, the intracellular signaling domain comprises a primary signaling sequence and a co-stimulatory signaling sequence. In some embodiments, the primary signaling sequence comprises a CD3ζ intracellular signaling sequence. In some embodiments, the co-stimulatory signaling sequence comprises a CD28 intracellular signaling sequence. In some embodiments, the intracellular domain comprises a CD3ζ intracellular signaling sequence and a CD28 intracellular signaling sequence.

### Anti-LAB TCR

In some embodiments, the anti-LAB antibody moiety is fused to the N-terminus of a T-cell receptor (TCR) subunit thus forming an anti-LAB T-cell receptor (herein referred to as an “anti-LAB TCR”).

“T cell receptor” or “TCR” as used herein refers to endogenous or recombinant T cell receptor comprising an extracellular antigen binding domain that binds to a specific antigenic peptide bound in an MHC molecule. In some embodiments, the TCR comprises a TCRα polypeptide chain and a TCRβ polypeptide chain. In some embodiments, the TCR comprises a TCRγ polypeptide chain and a TCRδ polypeptide chain. In some embodiments, the TCR specifically binds a tumor antigen. “TCR-T” refers to a T cell that expresses a recombinant TCR. “TCR complex” as used herein refers to a complex of TCR and CD3. “TCR subunits” used herein refers to a subunit of the TCR complex, which include, for example, TCRα, TCRβ, TCRγ, TCRδ, CD3ε, CD3γ, CD3δ, and CD3ζ. The anti-LAB antibody moiety can be fused to the N-terminus of any of the TCR subunits.

Thus, for example, in some embodiments, there is provided an engineered TCR, comprising: (a) an anti-LAB antibody moiety that specifically binds to Labyrinthin, and (b) a TCR subunit. In some embodiments, the antibody moiety is fused to the N-terminus of the TCR subunit. In some embodiments, the TCR subunit is selected from the group consisting of TCRα, TCRβ, TCRγ, TCRδ, CD3ε, CD3γ, CD3δ, and CD3ζ.

### Anti-Labyrinthin Bispecific Antibody

In some embodiments, the anti-LAB construct is a multi-specific antibody. Antibodies or antigen-binding fragments thereof having different specificities can also be chemically cross-linked to generate multi-specific heteroconjugate antibodies. For example, two F(ab′)2 molecules, each having specificity for a different antigen, can be chemically linked.

In some embodiments, the antibody moiety is multispecific. In some embodiments, the multispecific antibody moiety is a tandem scFv, a diabody (db), a single chain diabody (scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody. In some embodiments, the multispecific antibody moiety is a tandem scFv comprising two scFvs linked by an optional peptide linker.

In some embodiments, multi-specific antibodies can be prepared using recombinant DNA techniques. For example, a bispecific antibody can be engineered by fusing two scFvs, such as by fusing them through a peptide linker, resulting in a tandem scFv. One example of a tandem scFv is a bispecific T cell engager. Bispecific T cell engagers are made by linking an anti-CD3 scFv to an scFv specific for a surface antigen of a target cell, such as a tumor-associated antigen (TAA), resulting in the redirection of T cells to the target cells.

Thus, for example, in some embodiments, there is provided a bispecific T cell engager, comprising: (a) an anti-LAB antibody moiety that specifically binds to Labyrinthin, and (b) an anti-CD3 antibody moiety that specifically binds to CD3. In some embodiments, there is provided a bispecific T cell engager, comprising: (a) a scFv that specifically binds to Labyrinthin, and b) a scFv that specifically binds to CD3.

### Anti-LAB Antibody-Drug Conjugate (ADC) or Other Conjugates

The anti-LAB constructs, in some embodiments, comprise an immunoconjugate comprising an anti-LAB antibody moiety attached to an effector molecule (also referred to herein as an “anti-LAB immunoconjugate”). In some embodiments the effector molecule is a therapeutic agent, such as a cancer therapeutic agent, which is either cytotoxic, cytostatic or otherwise provides some therapeutic benefit. In some embodiments, the effector molecule is a label, which can generate a detectable signal, either directly or indirectly.

In some embodiments, there is provided an anti-LAB immunoconjugate comprising an anti-LAB antibody moiety and a therapeutic agent (also referred to herein as an “antibody-drug conjugate”, or “ADC”). In some embodiments, the therapeutic agent is a toxin that is either cytotoxic, cytostatic or otherwise prevents or reduces the ability of the target cells to divide. Importantly, since most normal cells do not present the LAB on their surface, they cannot bind the anti-LAB immunoconjugate, and are protected from the killing effect of the toxin or other therapeutic agents.

Therapeutic agents used in anti-LAB immunoconjugates include, for example, daunomycin, doxorubicin, methotrexate, and vindesine (see, e.g., Rowland et al., Cancer Immunol Immunother, 21, 1986). Toxins used in anti-LAB immunoconjugates include bacterial toxins such as diphtheria toxin, plant toxins such as ricin, small molecule toxins such as geldanamycin, maytansinoids, and calicheamicin. The toxins may exert their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

In some embodiments, the anti-LAB antibody moiety can be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

The present application further provides anti-LAB immunoconjugates comprising an anti-LAB antibody moiety attached to an effector molecule, wherein the effector molecule is a label, which can generate a detectable signal, indirectly or directly. These anti-LAB immunoconjugates can be used for research or diagnostic applications, such as for the in vivo detection of cancer. The label is preferably capable of producing, either directly or indirectly, a detectable signal. For example, the label may be radio-opaque or a radioisotope, such as 3H, 14C, 32P, 35S, 123I, 125I, 131I; a fluorescent (fluorophore) or chemiluminescent (chromophore) compound, such as fluorescein isothiocyanate, rhodamine or luciferin; an enzyme, such as alkaline phosphatase, β-galactosidase or horseradish peroxidase; an imaging agent; or a metal ion. In some embodiments, the anti-LAB immunoconjugate is detectable indirectly. For example, a secondary antibody that is specific for the anti-LAB immunoconjugate and contains a detectable label can be used to detect the anti-LAB immunoconjugate.

Thus, for example, in some embodiments, there is provided an anti-LAB immunoconjugate comprising: (a) an anti-LAB antibody moiety that specifically binds to Labyrinthin; and (b) an effector molecule. In some embodiments, the effector molecule is a therapeutic agent. In some embodiments, the effector molecule is a label.

### Anti-LAB Antibody Moieties

The present application in one aspect provides an antibody moiety that specifically binds to Labyrinthin or a portion thereof (herein referred to as “anti-LAB antibody moiety”). The anti-Labyrinthin constructs comprise an anti-LAB antibody moiety. In some embodiments, the anti-LAB antibody moiety specifically binds to Labyrinthin present on the surface of a cell. In some embodiments, the cell is a cancer cell. In some embodiments, the cancer cell is in a solid tumor, such as a metastatic cancer cell.

In some embodiments, the anti-LAB antibody moiety is a full-length antibody. In some embodiments, the anti-LAB antibody moiety is an antigen-binding fragment, for example, an antigen-binding fragment selected from the group consisting of a Fab, a Fab′, a F(ab′)2, an Fv fragment, a disulfide stabilized Fv fragment (dsFv), and a single-chain antibody molecule (scFv). In some embodiments, the anti-LAB antibody moiety is a scFv. In some embodiments, the anti-LAB antibody moiety is human, humanized, or semi-synthetic.

In some embodiments, the anti-LAB antibody moiety specifically binds to a Labyrinthin-derived peptide or a portion thereof. In some embodiments, the Labyrinthin-derived peptide comprises an amino acid sequence selected from a portion of Labyrinthin (SEQ ID NO:1: see Table 2). In some embodiments, the anti-LAB antibody moiety is derived from an antibody produced by using a Labyrinthin-derived peptide as an immunogen. In some embodiments, when the Labyrinthin-derived peptide is used as an immunogen, the Labyrinthin-derived peptide is conjugated to a carrier, such as albumin or KLH.

In some embodiments, the Labyrinthin-derived peptide is a peptide having a sequence similarity of at least about 60% similarity, such as at least about any of 65% similarity, 70% similarity, 75% similarity, 80% similarity, 85% similarity, 90% similarity, or 95% similarity, to a portion of Labyrinthin (SEQ ID NO:1), wherein the Labyrinthin-derived peptide does not comprise a terminal proline residue, and wherein the Labyrinthin-derived peptide comprises at least one proline residue, such as 2, 3, 4, or 5 proline residues. In some embodiments, the Labyrinthin-derived peptide is a peptide having a sequence similarity of at least about 60% similarity, such as at least about any of 65% similarity, 70% similarity, 75% similarity, 80% similarity, 85% similarity, 90% similarity, or 95% similarity, to a portion of Labyrinthin (SEQ ID NO:1), wherein one terminus of the Labyrinthin-derived peptide does not comprise a terminal proline residue, and wherein the Labyrinthin-derived peptide comprises at least one proline residue, such as 2, 3, 4, or 5 proline residues. In some embodiments, the Labyrinthin-derived peptide is between 7 and 50 amino acids in length, such as between any of 7 and 25 amino acids in length, 7 to 13 amino acids in length, or 21 and 25 amino acids in length. In some embodiments, the Labyrinthin-derived peptide is 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some embodiments, the Labyrinthin-derived peptide is substantially homologous to a portion of Labyrinthin (SEQ ID NO: 1). In some embodiments, the Labyrinthin-derived peptide has a sequence similarity of at least about 60% similarity to a portion of Labyrinthin (SEQ ID NO:1), such as at least about any of 65% similarity, 70% similarity, 75% similarity, 80% similarity, 85% similarity, 90% similarity, or 95% similarity. In some embodiments, the Labyrinthin-derived peptide or a derivative thereof has a sequence similarity of at least about 60% similarity to a portion of Labyrinthin (SEQ ID NO:1), wherein 1, 2, 3, 4, of 5 amino acids of the sequence of the Labyrinthin-derived peptide are deleted, substituted, inserted, and/or added to the Labyrinthin-derived peptide, and wherein when a substitution, insertion, and/or addition is present, a moiety is substituted, inserted, and/or added to the sequence. In some embodiments, the moiety that is substituted, inserted, or added is a natural amino acid (e.g., α-amino acid or a L-amino acid or a D-amino acid) or a non-natural amino acid. In some embodiments, the moiety that is substituted, inserted, or added is an amino acid substitute or a linker. In some embodiments, the Labyrinthin-derived peptide comprises a T-cell epitope and/or a B-cell epitope.

In some embodiments, the Labyrinthin-derived peptide comprises a sequence selected from SEQ ID NO:2-32 (Table 2), or variant thereof. In some embodiments, the Labyrinthin-derived peptide comprising a sequence selected from SEQ ID NO:2-32 (Table 2), or variant thereof, comprises one or two flanking amino acid sequences, the one or two flanking amino acid sequences added on the terminal ends of the core sequences provided in SEQ ID NO:2-32. In some embodiments, the Labyrinthin-derived peptide comprising one or two flanking amino acid sequences is a peptide having a sequence similarity of at least about 60% similarity, such as at least about any of 65% similarity, 70% similarity, 75% similarity, 80% similarity, 85% similarity, 90% similarity, or 95% similarity, to a portion of Labyrinthin (SEQ ID NO:1). In some embodiments, the flanking amino acid sequence is based on the sequence of a portion of Labyrinthin (SEQ ID NO:1) and is a respective continuation of the Labyrinthin sequence from each core sequence provided in SEQ ID NO:2-32, such as from one or two terminal ends of the core sequence. For example, for SEQ ID NO:3, a first flanking amino acid sequence of two amino acids to the left of SEQ ID NO:3 would be Pro-Ala, and a second flanking amino acid sequence of two amino acids to the right of SEQ ID NO:3 would be Glu-Ala.

TABLE 2
Amino acid sequences of Labyrinthin and Labyrinthin-derived peptides.

| | Amino acid sequence |
|---|---|
| SEQ ID NO: 1 | Met-Val-Ile-Ala-Leu-Leu-Gly-Val-Trp-Thr-Ser-Val-Ala-Val-Val-Trp-Phe- |
| | Asp-Leu-Val-Asp-Tyr-Glu-Glu-Val-Leu-Gly-Lys-Leu-Gly-Ile-Tyr-Asp-Ala- |
| | Asp-Gly-Asp-Gly-Asp-Phe-Asp-Val-Asp-Asp-Ala-Lys-Val-Leu-Leu-Gly- |
| | Leu-Lys-Glu-Arg-Ser-Thr-Ser-Glu-Pro-Ala-Val-Pro-Pro-Glu-Glu-Ala-Glu- |
| | Pro-His-Thr-Glu-Pro-Glu-Glu-Gln-Val-Pro-Val-Glu-Ala-Glu-Pro-Gln-Asn- |
| | Ile-Glu-Asp-Glu-Ala-Lys-Glu-Gln-Ile-Gln-Ser-Leu-Leu-His-Glu-Met-Val- |
| | His-Ala-Glu-His-Val-Glu-Gly-Glu-Asp-Leu-Gln-Gln-Glu-Asp-Gly-Pro-Thr- |
| | Gly-Glu-Pro-Gln-Gln-Glu-Asp-Asp-Glu-Phe-Leu-Met-Ala-Thr-Asp-Val- |
| | Asp-Asp-Arg-Phe-Glu-Thr-Leu-Glu-Pro-Glu-Val-Ser-His-Glu-Glu-Thr-Glu- |
| | His-Ser-Tyr-His-Val-Glu-Glu-Thr-Val-Ser-Gln-Asp-Cys-Asn-Gln-Asp-Met- |
| | Glu-Glu-Met-Met-Ser-Glu-Gln-Glu-Asn-Pro-Asp-Ser-Ser-Glu-Pro-Val-Val- |
| | Glu-Asp-Glu-Arg-Leu-His-His-Asp-Thr-Asp-Asp-Val-Thr-Tyr-Gln-Val- |
| | Tyr-Glu-Glu-Gln-Ala-Val-Tyr-Glu-Pro-Leu-Glu-Asn-Glu-Gly-Ile-Glu-Ile- |
| | Thr-Glu-Val-Thr-Ala-Pro-Pro-Glu-Asp-Asn-Pro-Val-Glu-Asp-Ser-Gln-Val- |
| | Ile-Val-Glu-Glu-Val-Ser-Ile-Phe-Pro-Val-Glu-Glu-Gln-Gln-Glu-Val-Pro- |
| | Pro-Asp-Thr |
| SEQ ID NO: 2 | Glu-Pro-Ala |
| SEQ ID NO: 3 | Val-Pro-Pro-Glu |
| SEQ ID NO: 4 | Glu-Pro-His |
| SEQ ID NO: 5 | Glu-Pro-Glu |
| SEQ ID NO: 6 | Val-Pro-Val |
| SEQ ID NO: 7 | Glu-Pro-Gln |
| SEQ ID NO: 8 | Gly-Pro-Thr |
| SEQ ID NO: 9 | Asn-Pro-Asp |
| SEQ ID NO: 10 | Glu-Pro-Val |
| SEQ ID NO: 11 | Glu-Pro-Leu |
| SEQ ID NO: 12 | Ala-Pro-Pro-Glu |
| SEQ ID NO: 13 | Asn-Pro-Val |
| SEQ ID NO: 14 | Phe-Pro-Val |
| SEQ ID NO: 15 | Val-Pro-Pro-Asp |
| SEQ ID NO: 16 | Glu-Pro-Ala-Val-Pro-Pro-Glu |
| SEQ ID NO: 17 | Val-Pro-Pro-Glu-Glu-Ala-Glu-Pro-His |
| SEQ ID NO: 18 | Glu-Pro-His-Thr-Glu-Pro-Glu |
| SEQ ID NO: 19 | Glu-Pro-Glu-Glu-Gln-Val-Pro-Val |
| SEQ ID NO: 20 | Val-Pro-Val-Glu-Ala-Glu-Pro-Gln |
| SEQ ID NO: 21 | Gly-Pro-Thr-Gly-Glu-Pro-Gln |
| SEQ ID NO: 22 | Asn-Pro-Asp-Ser-Ser-Glu-Pro-Val |
| SEQ ID NO: 23 | Ala-Pro-Pro-Glu-Asp-Asn-Pro-Val |
| SEQ ID NO: 24 | Phe-Pro-Val-Glu-Glu-Gln-Gln-Glu-Val-Pro-Pro-Asp |
| SEQ ID NO: 25 | Asp-Gly-Pro-Thr-Gly-Glu-Pro-Gln-Gln-Glu |
| SEQ ID NO: 26 | Glu-Gln-Glu-Asn-Pro-Asp-Ser-Ser-Glu-Pro-Val |
| SEQ ID NO: 27 | Ala-Pro-Pro-Glu-Asp-Asn-Pro-Val-Glu-Asp |
| SEQ ID NO: 28 | Glu-Glu-Gln-Gln-Glu-Val-Pro-Pro-Asp |
| SEQ ID NO: 29 | Gly-Glu-Asp-Leu-Gln-Gln-Glu-Asp-Gly-Pro-Thr-Gly-Glu-Pro-Gln-Gln- |
| | Glu-Asp-Asp-Glu-Phe-Leu |
| SEQ ID NO: 30 | Asp-Met-Glu-Glu-Met-Met-Ser-Glu-Gln-Glu-Asn-Pro-Asp-Ser-Ser-Glu- |
| | Pro-Val-Val-Glu-Asp-Glu |
| SEQ ID NO: 31 | Asn-Glu-Gly-Ile-Glu-Ile-Thr-Glu-Val-Thr-Ala-Pro-Pro-Glu-Asp-Asn-Pro- |
| | Val-Glu-Asp-Ser-Gln |
| SEQ ID NO: 32 | Asp-Ser-Gln-Val-Ile-Val-Glu-Glu-Val-Ser-Ile-Phe-Pro-Val-Glu-Glu-Gln- |
| | Gln-Glu-Val-Pro-Pro-Asp |

In some embodiments, the anti-LAB antibody moiety (or the anti-LAB construct comprising the anti-LAB antibody moiety) binds to Labyrinthin or a portion thereof with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values).

In some embodiments, the anti-LAB antibody moiety (or the anti-LAB construct comprising the anti-LAB antibody moiety) binds to one or more, such as 2 or 3, epitopes of Labyrinthin or a portion thereof.

The anti-LAB antibody moieties in some embodiments comprise specific sequences or certain variants of such sequences. In some embodiments, the amino acid substitutions in the variant sequences do not substantially reduce the ability of the anti-LAB antibody moiety to bind Labyrinthin or a portion thereof. For example, alterations that do not substantially reduce Labyrinthin binding affinity may be made. Alterations that substantially improve Labyrinthin binding affinity or affect some other property, such as specificity and/or cross-reactivity with related variants of the Labyrinthin, are also contemplated.

Anti-Labyrinthin antibodies have been disclosed in the art, e.g., U.S. Pat. Nos. 6,166,176, 7,635,759, and International publication No. WO2011116014, the disclosures of each of which are hereby incorporated by reference in their entirety. In some embodiments, the anti-LAB antibody moiety comprises MCA44-3A6 or a portion thereof that binds to Labyrinthin. In some embodiments, the anti-LAB antibody moiety is MCA44-3A6. In some embodiments, the anti-LAB antibody moiety competes with MCA44-3A6 or a portion thereof that binds to Labyrinthin. In some embodiments, the anti-LAB antibody moiety comprises one or more CDRs from MCA44-3A6. In some embodiments, the anti-LAB antibody moiety comprises X373 or a portion thereof that binds to Labyrinthin. In some embodiments, the anti-LAB antibody moiety is X373. In some embodiments, the anti-LAB antibody moiety competes with X373 or a portion thereof that binds to Labyrinthin. In some embodiments, the anti-LAB antibody moiety comprises X509 or a portion thereof that binds to Labyrinthin. In some embodiments, the anti-LAB antibody moiety is X509. In some embodiments, the anti-LAB antibody moiety competes with X509 or a portion thereof that binds to Labyrinthin. In some embodiments, the anti-LAB antibody moiety comprises one or more of the variable heavy chain domain complementarity-determining regions (CDRs) recited in SEQ ID NO:33, SEQ ID NO:34, and SEQ ID NO:35. In some embodiments, the anti-LAB antibody moiety comprises one or more of the variable light chain domain complementarity-determining regions (CDRs) recited in SEQ ID NO:36, SEQ ID NO:37, and SEQ ID NO:38. In some embodiments, the anti-LAB antibody moiety comprises one or more of the variable heavy chain domain complementarity-determining regions (CDRs) recited in SEQ ID NO:33, SEQ ID NO:34, and SEQ ID NO:35, and one or more of the variable light chain domain complementarity-determining regions (CDRs) recited in SEQ ID NO:36, SEQ ID NO:37, and SEQ ID NO:38.

TABLE 3
CDR sequences.

| | CDR sequences |
|---|---|
| SEQ ID NO: 33 | Lys-Ser-Ser-Gln-Ser-Leu-Val-Tyr-Ser- |
| | Asp-Gly-Lys-Thr-Tyr-Leu-Asn |
| SEQ ID NO: 34 | Leu-Val-Ser-Lys-Leu-Asp-Ser |
| SEQ ID NO: 35 | Leu-Gln-Gly-Ser-His-Phe-Pro-Arg-Thr |
| SEQ ID NO: 36 | Gly-Phe-Thr-Phe-Ser-Arg-Tyr-Thr-Met- |
| | Ser |
| SEQ ID NO: 37 | Tyr-Ile-Ser-Asn-Gly-Gly-Arg-Ser-Ile- |
| | Tyr-Tyr-Ala-Asp-Ser-Val-Lys-Gly |
| SEQ ID NO: 38 | Ala-Met-Asp-Asn |

### Nucleic Acids and Effector Cells

Nucleic acid molecules encoding the anti-LAB constructs or anti-LAB antibody moieties are also provided herein. In some embodiments, there is provided a nucleic acid (or a set of nucleic acids) encoding a full-length anti-LAB antibody. In some embodiments, there is provided a nucleic acid (or a set of nucleic acids) encoding a multi-specific anti-LAB molecule (e.g., a multi-specific anti-LAB antibody, a bispecific anti-LAB antibody, or a bispecific T-cell engager anti-LAB antibody), or polypeptide portion thereof. In some embodiments, there is provided a nucleic acid (or a set of nucleic acids) encoding an anti-LAB antibody moiety. In some embodiments, there is provided a nucleic acid (or a set of nucleic acids) encoding an anti-LAB immunoconjugate, or polypeptide portion thereof.

The present application also includes variants to these nucleic acid sequences. For example, the variants include nucleotide sequences that hybridize to the nucleic acid sequences encoding the anti-LAB constructs or anti-LAB antibody moieties of the present application under at least moderately stringent hybridization conditions.

The present invention also provides vectors in which a nucleic acid of the present invention is inserted.

In brief summary, the expression of an anti-LAB construct (e.g., anti-LAB CAR) or polypeptide portion thereof by a natural or synthetic nucleic acid encoding the anti-LAB construct or polypeptide portion thereof can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acids of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter. MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, pi-galactosidase, chloramphenicol acetyl transferase, secreted alkaline phosphatase, or the green fluorescent protein gene (e.g., Ui-Tel et al., FEBS Letters, 479, 2000). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In some embodiments, there is provided an effector cell (such as a T cell) presenting on its surface an anti-LAB construct described herein. In some embodiments, the effector cell (such as a T cell) comprises on its surface an anti-LAB CAR polypeptide described herein. In some embodiments, the effector cell comprises a nucleic acid encoding the anti-LAB CAR polypeptide described herein, wherein the anti-LAB CAR polypeptides, or a portion thereof, expressed from the nucleic acid are localized on the surface of the effector cell. In some embodiments, the effector cell (such as a T cell) comprises on its surface an anti-LAB TCR polypeptide described herein. In some embodiments, the effector cell comprises a nucleic acid encoding the anti-LAB TCR polypeptide described herein, wherein the anti-LAB TCR polypeptides, or a portion thereof, expressed from the nucleic acid are localized on the surface of the effector cell. In some embodiments, the effector cell (such as a T cell) comprises on its surface a multi-specific antibody, such as a bispecific antibody, polypeptide described herein. In some embodiments, the effector cell comprises a nucleic acid encoding the multi-specific antibody, such as a bispecific antibody, polypeptide described herein, wherein the multi-specific antibody, such as a bispecific antibody, polypeptides, or a portion thereof, expressed from the nucleic acid are localized on the surface of the effector cell.

Exemplary effector cells useful for the present invention include, but are not limited to, dendritic cells (including immature dendritic cells and mature dendritic cells), T lymphocytes (such as naïve T cells, effector T cells, memory T cells, cytotoxic T lymphocytes, T helper cells, Natural Killer T cells, Treg cells, tumor infiltrating lymphocytes (TIL), and lyphokine-activated killer (LAK) cells), B cells, Natural Killer (NK) cells, monocytes, macrophages, neutrophils, granulocytes, and combinations thereof. Subpopulations of effector cells can be defined by the presence or absence of one or more cell surface markers known in the art (e.g., CD3, CD4, CD8, CD19, CD20, CD11c, CD123, CD56, CD34, CD14, CD33, etc.).

In some embodiments, the effector cell is an engineer effector cell, such as an engineered mammalian effector cell. In the cases that the pharmaceutical composition comprises a plurality of engineered mammalian effector cells, the engineered mammalian effector cells can be a specific subpopulation of an effector cell type, a combination of subpopulations of an effector cell type, or a combination of two or more effector cell types. In some embodiments, the effector cell is present in a homogenous cell population. In some embodiments, the effector cell is present in a heterogeneous cell population that is enhanced in the effector cell. In some embodiments, the engineered mammalian cell is a lymphocyte. In some embodiments, the engineered mammalian cell is not a lymphocyte. In some embodiments, the engineered mammalian cell is suitable for adoptive immunotherapy. In some embodiments, the engineered mammalian cell is a PBMC. In some embodiments, the engineered mammalian cell is an effector cell derived from the PBMC. In some embodiments, the engineered mammalian cell is a T cell. In some embodiments, the engineered mammalian cell is a CD4+ T cell. In some embodiments, the engineered mammalian cell is a CD8+ T cell. In some embodiments, the engineered mammalian cell is a B cell. In some embodiments, the engineered mammalian cell is an NK cell.

In some embodiments, the effector cell is a T cell. In some embodiments, the effector cell is selected from the group consisting of a cytotoxic T cell, a helper T cell, a natural killer T cell, and a suppressor T cell. In some embodiments, the effector cell is modified to block or decrease the expression of one or both of the endogenous TCR subunits from which the chimeric receptor polypeptides are derived. Modifications of cells to disrupt gene expression include any such techniques known in the art, including for example RNA interference (e.g., siRNA, shRNA, miRNA), gene editing (e.g., CRISPR- or TALEN-based gene knockout), and the like.

### Methods of Making Anti-LAB and Body Moieties and Constructs Thereof

The present application in one aspect provides methods of making anti-LAB antibody moieties and constructs thereof.

### Monoclonal Antibodies

In some embodiments, the anti-LAB antibody moiety or construct thereof comprises a monoclonal antibody. Monoclonal antibodies can be prepared, e.g., using hybridoma methods, such as those described by Kohler and Milstein, Nature, 256, 1975 and Sergeeva et al., Blood, 117(16):4262-4272, using the phage display methods described herein and in the Examples below, or using recombinant DNA methods (see, e.g., U.S. Pat. No. 4,816,567).

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest, or a complex comprising at least two molecules. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. See. e.g., Goding, Monoclonal Antibodies: Principles and Practice (New York: Academic Press, 1986). Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. Kozbor, J. Immunol., 133:3001 (1984); Brodeur et al. Monoclonal Antibody Production Techniques and Applications (Marcel Dekker, Inc.: New York, 1987) pp. 51-63.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be sub cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The anti-LAB antibody moieties may also be identified by screening combinatorial libraries for antibodies with the desired activity or activities. For example, a variety of methods are known in the art for generating phage display libraries and screening such libraries for antibodies possessing the desired binding characteristics. Such methods arm reviewed, e.g., in Hoogenboom et al. Methods in Molecular Biology 178:1-37 (O'Brien et al. ed., Human Press, Totowa, N.J., 2001) and further described, e.g., in McCafferty et al., Nature 348:552-554; Clackson et al., Nature 352: 624-628 (1991); Marks e al., J. Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J. 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The antibodies or antigen-binding fragments thereof can be prepared using phage display to screen libraries for antibodies specific to Labyrinthin or a portion thereof. The library can be a human scFv phage display library having a diversity of at least one×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see. e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a non-human phage display library.

Phage clones that bind to Labyrinthin or a portion thereof with high affinity can be selected by iterative binding of phage to Labyrinthin, which is bound to a solid support (such as, for example, beads for solution panning or mammalian cells for cell panning), followed by removal of non-bound phage and by elution of specifically bound phage. In an example of solution panning, Labyrinthin can be biotinylated for immobilization to a solid support. The biotinylated Labyrinthin is mixed with the phage library and a solid support, such as streptavidin-conjugated Dynabeads M-280, and then Labyrinthin-phage-bead complexes are isolated. The bound phage clones are then cluted and used to infect an appropriate host cell, such as E. coli XL I-Blue, for expression and purification. The panning can be performed for multiple (such as about any of 2, 3, 4, 5, 6 or more) rounds with either solution panning, cell panning, or a combination of both, to enrich for phage clones binding specifically to Labyrinthin. Enriched phage clones can be tested for specific binding to Labyrinthin by any methods known in the art, including for example ELISA and FACS.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). Hybridoma cells as described above or Labyrinthin-specific phage clones of the invention can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains and/or framework regions in place of the homologous non-human sequences (U.S. Pat. No. 4,816,567; Morrison et al., supra) or by covalently joining to the immunoglobulin coding sequence all or part of the coding sequence for a nonimmunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using any method known in the art.

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. In some embodiments, the first heavy-chain constant region (CH1) containing the site necessary for light-chain binding is present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies, see. for example, Suresh et al., Methods in Enzymology, 121: 210 (1986).

### Human and Humanized Antibodies

In some embodiments, the anti-LAB antibody moiety or construct thereof comprises a humanized antibodies or human antibodies. Humanized forms of non-human (e.g., murine) antibodies are chimeric immunoglobulins, immunoglobulin chains, or fragments thereof (such as Fv, Fab, Fab′, F(ab′)₂, scFv, or other antigen-binding subsequences of antibodies) that typically contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody can comprise substantially all of at least one, and typically two, variable domains, in which all or substantially all of the CDR regions correspond to those of a non-human immunoglobulin, and all or substantially all of the FR regions are those of a human immunoglobulin consensus sequence. In some embodiments, the humanized antibody will comprise at least a portion of an immunoglobulin constant region (Fc), typically that of a human immunoglobulin. See, e.g., Jones et al., Nature, 321: 522-525 (1986); Riechmann et al., Nature, 332: 323-329 (1988); Presta, Curr. Op. Struct. Biol., 2:593-596 (1992).

Generally, a humanized antibody has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibodies are antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993): U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994): Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Human antibodies may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

### Multi-Specific Antibodies

In some embodiments, the anti-LAB antibody moiety or construct thereof comprises a multi-specific antibody. Suitable methods for making multi-specific (e.g., bispecific) antibodies are well known in the art. For example, the production of bispecific antibodies can be based on the co-expression of two immunoglobulin heavy-chain/light-chain pairs, where the two pairs each have different specificities, and upon association result in a heterodimeric antibody (see, e.g., Milstein and Cuello, Nature, 305: 537-539 (1983); WO 93/08829, and Traunecker et al., EMBO J. 10: 3655 (1991)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829 and in Traunecker et al., EMBO, 10: 3655-3659 (1991). Alternatively, the combining of heavy and light chains can be directed by taking advantage of species-restricted pairing (see, e.g., Lindhofer et al., J. Immunol., 155:219-225 (1995)) and the pairing of heavy chains can be directed by use of “knob-into hole” engineering of CH3 domains (see, e.g., U.S. Pat. No. 5,731,168; Ridgway et al., Protein Eng., 9(7):617-621 (1996)). Multi-specific antibodies may also be made by engineering electrostatic steering effects for making antibody Fc-heterodimeric molecules (see, e.g., WO 2009/089004A1). In yet another method, stable bispecific antibodies can be generated by controlled Fab-arm exchange, where two parental antibodies having distinct antigen specificity and matched point mutations in the CH3 domains are mixed in reducing condition to allow for separation, reassembly, and reoxidation to form highly pure bispecific antibodies. Labrigin et al., Proc. Natl. Acad. Sci., 110(13):5145-5150 (2013). Such antibodies, comprising a mixture of heavy-chain/light-chain pairs, are also referred to herein as “heteromultimeric antibodies”.

Antibodies or antigen-binding fragments thereof having different specificities can also be chemically cross-linked to generate multi-specific heteroconjugate antibodies. For example, two F(ab′)2 molecules, each having specificity for a different antigen, can be chemically linked. Pullarkat et al., Trends Biotechnol., 48:9-21 (1999). Such antibodies have, for example, been proposed to target immune-system cells to unwanted cells (U.S. Pat. No. 4,676,980), and for treatment of HIV infection. WO 91/00360: WO 92/200373: EP 03089. It is contemplated that the antibodies can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolate and methyl-4-mercaptobutyrimidate and those disclosed, for example, in U.S. Pat. No. 4,676,980.

In some embodiments, multi-specific antibodies can be prepared using recombinant DNA techniques. For example, a bispecific antibody can be engineered by fusing two scFvs, such as by fusing them through a peptide linker, resulting in a tandem scFv. One example of a tandem scFv is a bispecific T cell engager. Bispecific T cell engagers are made by linking an anti-CD3 scFv to an scFv specific for a surface antigen of a target cell, such as a tumor-associated antigen (TAA), resulting in the redirection of T cells to the target cells. Mack et al., Proc. Natl. Acad. Sci., 92:7021-7025 (1995): Brischwein et al., Mol. Immunol., 43(8):1129-1143 (2006). By shortening the length of a peptide linker between two variable domains, they can be prevented from self-assembling and forced to pair with domains on a second polypeptide, resulting in a compact bispecific antibody called a diabody (db). Holliger et al., Proc. Natl. Acad. Sci., 90:6444-6448 (1993). The two polypeptides of a db each comprise a VH connected to a VL by a linker which is too short to allow pairing between the two domains on the same chain. Accordingly, the VH and VL domains of one polypeptide are forced to pair with the complementary VL and VH domains of another polypeptide, thereby forming two antigen-binding sites. In a modification of this format, the two polypeptides are linked by another peptide linker, resulting in a single chain diabody (scDb). In yet another modification of the db format, dual-affinity retargeting (DART) bispecific antibodies can be generated by introducing a disulfide linkage between cysteine residues at the C-terminus of each polypeptide, optionally including domains prior to the C-terminal cysteine residues that drive assembly of the desired heterodimeric structure. Veri et al., Arthritis Rheum., 62(7):1933-1943 (2010). Dual-variable-domain immunoglobulins (DVD-Ig™), in which the target-binding variable domains of two monoclonal antibodies are combined via naturally occurring linkers to yield a tetravalent, bispecific antibody, are also known in the art. Gu and Ghayur, Methods Enzymol., 502:2541 (2012). In yet another format, Dock and Lock (DNL), bispecific antibodies are prepared by taking advantage of the dimerization of a peptide (DDD2) derived from the regulatory subunit of human cAMP-dependent protein kinase (PKA) with a peptide (AD2) derived from the anchoring domains of human A kinase anchor proteins (AKAPs). Rossi et al., Proc. Natl. Acad. Sci., 103:6841-6846 (2006).

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). This method can also be utilized for the production of antibody homodimers.

### Anti-LAB Antibody Moiety Variants

In some embodiments, amino acid sequence variants of the antibody moieties provided herein are contemplated. For example, it may be desirable to improve the binding affinity and/or other biological properties of the antibody moiety. Amino acid sequence variants of an antibody moiety may be prepared by introducing appropriate modifications into the nucleotide sequence encoding the antibody moiety, or by peptide synthesis. Such modifications include, for example, deletions from, and/or insertions into and/or substitutions of residues within the amino acid sequences of the antibody moiety. Any combination of deletion, insertion, and substitution can be made to arrive at the final construct, provided that the final construct possesses the desired characteristics, e.g., antigen-binding.

In some embodiments, antibody moiety variants having one or more amino acid substitutions are provided. Sites of interest for substitutional mutagenesis include the HVRs and FRs. Amino acid substitutions may be introduced into an antibody moiety of interest and the products screened for a desired activity, e.g., retained/improved antigen binding, decreased immunogenicity, or improved ADCC or CDC.

Conservative substitutions are shown in Table 4 below.

TABLE 4
CONSERVATIVE SUBSTITITIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, lie;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

An exemplary substitutional variant is an affinity matured antibody moiety, which may be conveniently generated, e.g., using phage display-based affinity maturation techniques. Briefly, one or more CDR residues are mutated and the variant antibody moieties displayed on phage and screened for a particular biological activity (e.g. binding affinity). Alterations (e.g., substitutions) may be made in HVRs, e.g., to improve antibody moiety affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or specificity determining residues (SDRs), with the resulting variant VH or VL being tested for binding affinity. Affinity maturation by constructing and reselecting from secondary libraries has been described. e.g., in Hoogenboom et al. in Methods in Molecular Biology 178:1-37 (O'Brien et al., ed., Human Press, Totowa, N.J., (2001).)

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody moiety variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody moiety to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant VH and VL sequences provided above, each HVR either is unaltered, or contains no more than one, two or three amino acid substitutions.

A useful method for identification of residues or regions of an antibody moiety that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody moiety with antigen is affected. Further substitutions may be introduced at the amino acid locations demonstrating functional sensitivity to the initial substitutions. Alternatively, or additionally, a crystal structure of an antigen-antibody moiety complex can be determined to identify contact points between the antibody moiety and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody moiety with an N-terminal methionyl residue. Other insertional variants of the antibody moiety include the fusion to the N- or C-terminus of the antibody moiety to an enzyme (e.g. for ADEPT) or a polypeptide which increases the serum half-life of the antibody moiety.

### Fc Region Variants

In some embodiments, one or more amino acid modifications may be introduced into the Fc region of a full-length anti-LAB antibody moiety provided herein, thereby generating an Fc region variant. In some embodiments, the Fc region variant has enhanced antibody dependent cellular cytotoxicity (ADCC) effector function, often related to binding to Fc receptors (FcRs). In some embodiments, the Fc region variant has decreased ADCC effector function. There are many examples of changes or mutations to Fc sequences that can alter effector function. For example, WO 00/42072 and Shields et al. J Biol. Chem. 9(2): 6591-6604 (2001) describe antibody variants with improved or diminished binding to FcRs. The contents of those publications are specifically incorporated herein by reference.

Antibody-Dependent Cell-Mediated Cytotoxicity (ADCC) is a mechanism of action of therapeutic antibodies against tumor cells. ADCC is a cell-mediated immune defense whereby an effector cell of the immune system actively lyses a target cell (e.g., a cancer cell), whose membrane-surface antigens have been bound by specific antibodies (e.g., an anti-LAB antibody). The typical ADCC involves activation of NK cells by antibodies. NK cells express CD16 which is an Fc receptor. This receptor recognizes, and binds to, the Fc portion of an antibody bound to the surface of a target cell. The most common Fc receptor on the surface of NK cells is called CD16 or FcγRIII. Binding of the Fc receptor to the Fc region of an antibody results in NK cell activation, release of cytolytic granules and consequent target cell apoptosis. The contribution of ADCC to tumor cell killing can be measured with a specific test that uses NK-92 cells that have been transfected with a high-affinity FcR. Results are compared to wild-type NK-92 cells that do not express the FcR.

In some embodiments, the invention contemplates an anti-LAB construct variant comprising an FC region that possesses some but not all effector functions, which makes it a desirable candidate for applications in which the half-life of the anti-LAB construct in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, there is provided an anti-LAB construct (e.g., a full-length anti-LAB antibody) variant comprising a variant Fc region comprising one or more amino acid substitutions which improve ADCC. In some embodiments, the variant Fc region comprises one or more amino acid substitutions which improve ADCC, wherein the substitutions are at positions 298, 333, and/or 334 of the variant Fc region (EU numbering of residues). In some embodiments, the anti-LAB construct (e.g., full-length anti-LAB antibody) variant comprises the following amino acid substitution in its variant Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551. WO 99/51642, and Idusogie et al, J. Immunol. 164: 4178-4184 (2000).

In some embodiments, there is provided an anti-LAB construct (e.g., a full-length anti-LAB antibody) variant comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988): U.S. Pat. Nos. 5,648,260; 5,624,821; and WO 94/29351 concerning other examples of Fc region variants.

Anti-LAB constructs (such as full-length anti-LAB antibodies) comprising any of the Fc variants described herein, or combinations thereof, are contemplated.

### Glycosylation Variants

In some embodiments, an anti-LAB construct provided herein is altered to increase or decrease the extent to which the anti-LAB construct is glycosylated. Addition or deletion of glycosylation sites to an anti-LAB construct may be conveniently accomplished by altering the amino acid sequence of the anti-LAB construct or polypeptide portion thereof such that one or more glycosylation sites is created or removed.

Where the anti-LAB construct comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-LAB construct of the invention may be made in order to create anti-LAB construct variants with certain improved properties.

In some embodiments, anti-LAB construct (such as full-length anti-LAB antibody) variants are provided comprising an Fc region wherein a carbohydrate structure attached to the Fc region has reduced fucose or lacks fucose, which may improve ADCC function. Specifically, anti-LAB constructs are contemplated herein that have reduced fucose relative to the amount of fucose on the same anti-LAB construct produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In some embodiments, the anti-LAB construct is one wherein less than about 50%, 40%, 30%, 20%, 10%, or 5% of the N-linked glycans thereon comprise fucose. For example, the amount of fucose in such an anti-LAB construct may be from 1% to 80%, from 1% to 65%, from 5% to 65% or from 20% to 40%. In some embodiments, the anti-LAB construct is one wherein none of the N-linked glycans thereon comprise fucose, i.e., wherein the anti-LAB construct is completely without fucose, or has no fucose or is afucosylated. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et 7. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al Arch. Biochem. Biophys. 249:533-545 (1986); US Pat Appl No US 2003/0157108 A1, Presta, L; and WO 2004/056312 A1, Adams et al., especially at Example 11), and knockout cell lines, such as α-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Anti-LAB construct (such as full-length anti-LAB antibody) variants are further provided with bisected oligosaccharides, e.g., in which a biantennary oligosaccharide attached to the Fc region of the anti-LAB construct is bisected by GlcNAc. Such anti-LAB construct (such as full-length anti-LAB antibody) variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Anti-LAB construct (such as full-length anti-LAB antibody) variants with at least one galactose residue in the oligosaccharide attached to the Fc region are also provided. Such anti-LAB construct variants may have improved CDC function. Such antibody variants are described, e.g., in WO 1997/30087 (Patel et al.); WO 1998/58964 (Raju, S.); and WO 1999/22764 (Raju, S.).

In some embodiments, the anti-LAB construct (such as full-length anti-LAB antibody) variants comprising an Fc region are capable of binding to an FcγRIII. In some embodiments, the anti-LAB construct (such as full-length anti-LAB antibody) variants comprising an Fc region have ADCC activity in the presence of human effector cells or have increased ADCC activity in the presence of human effector cells compared to the otherwise same anti-LAB construct (such as full-length anti-LAB antibody) comprising a human wild-type IgG1Fc region.

### Cysteine Engineered Variants

In some embodiments, it may be desirable to create cysteine engineered anti-LAB constructs (such as full-length anti-LAB antibodies) in which one or more amino acid residues are substituted with cysteine residues. In some embodiments, the substituted residues occur at accessible sites of the anti-LAB construct. By substituting those residues with cysteine, reactive thiol groups are thereby positioned at accessible sites of the anti-LAB construct and may be used to conjugate the anti-LAB construct to other moieties, such as drug moieties or linker-drug moieties, to create an anti-LAB immunoconjugate, as described further herein. Cysteine engineered anti-LAB constructs (such as full-length anti-LAB antibodies) may be generated as described, e.g., in U.S. Pat. No. 7,521,541.

### Derivatives

In some embodiments, an anti-LAB construct provided herein may be further modified to contain additional nonproteinaceous moieties that are known in the art and readily available. The moieties suitable for derivatization of the anti-LAB construct include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the anti-LAB construct may vary, and if more than one polymer is attached, they can be the same or different molecules. In general, the number and/or type of polymers used for derivatization can be determined based on considerations including, but not limited to, the particular properties or functions of the anti-LAB construct to be improved, whether the anti-LAB construct derivative will be used in a therapy under defined conditions, etc.

In some embodiments, conjugates of an anti-LAB construct and nonproteinaceous moiety that may be selectively heated by exposure to radiation are provided. In some embodiments, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the anti-LAB construct-nonproteinaceous moiety are killed.

### Anti-LAB CAR, Anti-LAB TCR, and Anti-LAB Bispecific T Cell Engager

The present application in one aspect provides anti-LAB constructs, wherein the anti-LAB construct is, e.g., an anti-LAB CAR, anti-LAB TCR, or anti-LAB bispecific T cell engager. Methods for making such anti-LAB constructs is known in the art, and is described. e.g., in the sections above.

### Effector Cell Preparation

The present invention in one aspect provides effector cells (such as lymphocytes, for example T cells) that expressed, e.g., an anti-LAB CAR. Exemplary methods of preparing effector cells (such as T cells) expressing the anti-LAB CARs (anti-LAB CAR effector cells, such as anti-LAB CAR T cells) are provided herein.

In some embodiments, an anti-LAB CAR effector cell (such as T cell) can be generated by introducing a vector (including for example a lentiviral vector) comprising an anti-LAB CAR (for example a CAR comprising an anti-LAB antibody moiety and CD28 and CD3ζ intracellular signaling sequences) into the effector cell (such as T cell). In some embodiments, the anti-LAB CAR effector cells (such as T cells) of the invention are able to replicate in vivo, resulting in long-term persistence that can lead to sustained control of a Labyrinthin-positive disease (such as cancer, e.g., adenocarcinoma).

In some embodiments, the invention relates to administering a genetically modified T cell expressing an anti-LAB CAR for the treatment of a patient having a Labyrinthin-positive disease or at risk of having a Labyrinthin-positive disease using lymphocyte infusion. In some embodiments, autologous lymphocyte infusion is used in the treatment. Autologous PBMCs are collected from a patient in need of treatment and T cells are activated and expanded using the methods described herein and known in the art and then infused back into the patient.

In some embodiments, the anti-LAB CAR T cell expresses an anti-LAB CAR comprising an anti-LAB antibody moiety (also referred to herein as an “anti-LAB CAR T cell”). In some embodiments, the anti-LAB CAR T cell expresses an anti-LAB CAR comprising an extracellular domain comprising an anti-LAB antibody moiety and an intracellular domain comprising intracellular signaling sequences of CD3ζ and CD28. The anti-LAB CAR T cells of the invention can undergo robust in vivo T cell expansion and can establish Labyrinthin-specific memory cells that persist at high levels for an extended amount of time in blood and bone marrow. In some embodiments, the anti-LAB CAR T cells of the invention infused into a patient can eliminate Labyrinthin-presenting cells, such as Labyrinthin-presenting cancer cells, in vivo in patients having a Labyrinthin-positive disease. In some embodiments, the anti-LAB CAR T cells of the invention infused into a patient can eliminate Labyrinthin-presenting cells, such as Labyrinthin-presenting cancer cells, in vivo in patients having a Labyrinthin-positive disease that is refractory to at least one conventional treatment.

Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments of the present invention, any number of T cell lines available in the art may be used. In some embodiments of the present invention, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as Ca⁺-free, Mg²⁺-free PBS, PlasmaLyte A, or other saline solutions with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least one, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such as in isolating tumor infiltrating lymphocytes (TIL) from tumor tissue or from immune-compromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In some embodiments, it may be desirable to enrich for or positively select for regulatory T cells which typically express CD4⁺, CD25⁺, CD62Lhi, GITR⁺, and FoxP3⁺. Alternatively, in some embodiments, T regulatory cells are depleted by anti-CD25 conjugated beads or other similar methods of selection.

For isolation of a desired population of cells by positive or negative selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/mi is used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In some embodiments of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in some embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Whether prior to or after genetic modification of the T cells to express a desirable anti-LAB CAR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514: 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

### Immunoconjugate Preparation

The anti-LAB immunoconjugates may be prepared using any methods known in the art. See, e.g., WO 2009/067800. WO 2011/133886, and U.S. Patent Application Publication No. 2014322129, incorporated by reference herein in their entirety.

The anti-LAB antibody moiety of an anti-LAB immunoconjugate may be “attached to” the effector molecule by any means by which the anti-LAB antibody moiety can be associated with, or linked to, the effector molecule. For example, the anti-LAB antibody moiety of an anti-LAB immunoconjugate may be attached to the effector molecule by chemical or recombinant means. Chemical means for preparing fusions or conjugates are known in the art and can be used to prepare the anti-LAB immunoconjugate. The method used to conjugate the anti-LAB antibody moiety and effector molecule must be capable of joining the binding protein with the effector molecule without interfering with the ability of the binding protein to bind to the antigen on the target cell.

The anti-LAB antibody moiety of an anti-LAB immunoconjugate may be linked indirectly to the effector molecule. For example, the anti-LAB antibody moiety of an anti-LAB immunoconjugate may be directly linked to a liposome containing the effector molecule of one of several types. The effector molecule(s) and/or the anti-LAB antibody moiety may also be bound to a solid surface.

In some embodiments, the anti-LAB antibody moiety of an anti-LAB immunoconjugate and the effector molecule are both proteins and can be conjugated using techniques well known in the art. There are several hundred crosslinkers available that can conjugate two proteins. (See for example “Chemistry of Protein Conjugation and Crosslinking” 1991, Shans Wong, CRC Press, Ann Arbor). The crosslinker is generally chosen based on the reactive functional groups available or inserted on the anti-LAB antibody moiety and/or effector molecule. In addition, if there are no reactive groups, a photoactivatable crosslinker can be used. In certain instances, it may be desirable to include a spacer between the anti-LAB antibody moiety and the effector molecule. Crosslinking agents known to the art include the homobifunctional agents: glutaraldehyde, dimethyladipimidate and Bis(diazobenzidine) and the heterobifunctional agents: m Maleimidobenzoyl-N-Hydroxysuccinimide and Sulfo-m Maleimidobenzoyl-N-Hydroxysuccinimide.

In some embodiments, the anti-LAB antibody moiety of an anti-LAB immunoconjugate may be engineered with specific residues for chemical attachment of the effector molecule. Specific residues used for chemical attachment of molecule known to the art include lysine and cysteine. The crosslinker is chosen based on the reactive functional groups inserted on the anti-LAB antibody moiety, and available on the effector molecule.

An anti-LAB immunoconjugate may also be prepared using recombinant DNA techniques. In such a case a DNA sequence encoding the anti-LAB antibody moiety is fused to a DNA sequence encoding the effector molecule, resulting in a chimeric DNA molecule. The chimeric DNA sequence is transfected into a host cell that expresses the fusion protein. The fusion protein can be recovered from the cell culture and purified using techniques known in the art.

Examples of attaching an effector molecule, which is a label, to the binding protein include the methods described in Hunter, et al., Nature 144:945 (1962); David, et al., Biochemistry 13:1014 (1974); Pain, et al., J. Immunol. Meth. 40:219 (1981); Nygren, J. Histochem. and Cytochem. 30:407 (1982); Wensel and Meares, Radioimmunoimaging And Radioimmunotherapy, Elsevier, N.Y. (1983); and Colcher et al. “Use Of Monoclonal Antibodies As Radiopharmaceuticals For The Localization Of Human Carcinoma Xenografts In Athymic Mice” Meth. Enzymol., 121:802-16 (1986).

The radio- or other labels may be incorporated in the immunoconjugate in known ways. For example, the peptide may be biosynthesized or may be synthesized by chemical amino acid synthesis using suitable amino acid precursors involving, for example, fluorine-19 in place of hydrogen. Labels such as ⁹⁹Tc or ¹²³I, ¹⁸⁶Re, ¹⁸⁸Re and ¹¹¹In can be attached via a cysteine residue in the peptide. Yttrium-90 can be attached via a lysine residue. The IODOGEN method (Fraker et al., Biochem. Biophys. Res. Commun. 80:49-57 (1978)) can be used to incorporate iodine-123. “Monoclonal Antibodies in Immunoscintigraphy” (Chatal, CRC Press 1989) describes other methods in detail.

Immunoconjugates of the antibody moiety and a cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCI), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene tnaminepentaacetic acid (MX-DTPA) is an exemplary chelating agent for conjugation of radionucleotide to the antibody. See, e.g., WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The anti-LAB immunoconjugates of the invention expressly contemplate, but are not limited to, ADC prepared with cross-linker reagents: BMPS, EMCS, GMBS, HBVS, LC-SMCC, NIBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A). See pages 467498, 2003-2004 Applications Handbook and Catalog.

### Pharmaceutical Compositions

Also provided herein are compositions (such as pharmaceutical compositions, also referred to herein as formulations) comprising an anti-LAB construct. In some embodiments, the composition further comprises a cell (such as an effector cell, e.g., a T cell) associated with the anti-LAB construct. In some embodiments, there is provided a pharmaceutical composition comprising an anti-LAB construct and a pharmaceutically acceptable carrier. In some embodiments, the pharmaceutical composition further comprises a cell (such as an effector cell, e.g., a T cell) associated with the anti-LAB construct.

Suitable formulations of the anti-LAB constructs are obtained by mixing an anti-LAB construct having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben: catechol: resorcinol; cyclohexanol; 3-pentanol; and m-cresol); low molecular weight (less than about 10 residues) polypeptides; proteins, such as scrum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins: chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™. PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be treated herein. Lipofectins or liposomes can be used to deliver the anti-LAB constructs of this invention into cells.

The formulation herein may also contain one or more active compounds in addition to the anti-LAB construct as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent in addition to the anti-LAB construct. Such molecules are suitably present in combination in amounts that are effective for the purpose intended. The effective amount of such other agents depends on the amount of anti-LAB construct present in the formulation, the type of disease or disorder or treatment, and other factors discussed above. These are generally used in the same dosages and with administration routes as described herein or about from 1 to 99% of the heretofore employed dosages.

The anti-LAB constructs may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Sustained-release preparations may be prepared.

Sustained-release preparations of the anti-LAB constructs can be prepared. Suitable examples of sustained-release preparations include semipermeable matrices of solid hydrophobic polymers containing the antibody (or fragment thereof), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydro gels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization of anti-LAB constructs depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

In some embodiments, the anti-LAB construct is formulated in a buffer comprising a citrate. NaCl, acetate, succinate, glycine, polysorbate 80 (Tween 80), or any combination of the foregoing. In some embodiments, the anti-LAB construct is formulated in a buffer comprising about 100 mM to about 150 mM glycine. In some embodiments, the anti-LAB construct is formulated in a buffer comprising about 50 mM to about 100 mM NaCl. In some embodiments, the anti-LAB construct is formulated in a buffer comprising about 10 mM to about 50 mM acetate. In some embodiments, the anti-LAB construct is formulated in a buffer comprising about 10 mM to about 50 mM succinate. In some embodiments, the anti-LAB construct is formulated in a buffer comprising about 0.005% to about 0.02% polysorbate 80. In some embodiments, the anti-LAB construct is formulated in a buffer having a pH between about 5.1 and 5.6. In some embodiments, the anti-LAB construct is formulated in a buffer comprising 10 mM citrate, 100 mM NaCl, 100 mM glycine, and 0.01% polysorbate 80, wherein the formulation is at pH 5.5. The formulations to be used for in vivo administration must be sterile. This is readily accomplished by, e.g., filtration through sterile filtration membranes.

### Methods of Using Anti-LAB Constructs

The anti-LAB constructs and/or compositions of the application can be administered to individuals (e.g., mammals such as humans) to treat and/or prevent a disease and/or disorder, including, for example, cancer (such as adenocarcinoma). In some embodiments, the cancer is a Labyrinthin-positive cancer. In some embodiments, the cancer is an adenocarcinoma, such as Labyrinthin-positive adenocarcinoma. The present application thus in some embodiments provides a method of treating an Labyrinthin-positive disease (such as cancer) in an individual comprising administering to the individual an effective amount of a composition (such as a pharmaceutical composition) comprising an anti-LAB construct comprising an anti-LAB antibody moiety, such as any one of the anti-LAB constructs described herein. In some embodiments, the composition comprises a cell (such as an effector cell) expressing the anti-LAB construct (such as CAR).

For example, in some embodiments, there is provided a method of treating a Labyrinthin-positive disease in an individual comprising administering to the individual an effective amount of a composition comprising an anti-LAB construct comprising an anti-LAB antibody moiety that specifically binds to Labyrinthin. In some embodiments, there is provided a method of treating a Labyrinthin-positive disease in an individual comprising administering to the individual an effective amount of a composition comprising an effector cell (such as immune cell, for example T cell) expressing an anti-LAB construct (e.g., anti-LAB CAR or anti-LAB TCR described herein). In some embodiments, the individual is selected for treatment when a cancer sample from the individual shows that about 10% or greater, such as at least about any of 15%, 20%, 25%, 30%, 35%, 40%, 45%, 50%, 55%, 60%, 65%, 75%, 80%, 85%, 90%, or 95%, of cancerous cells in the sample are positive for Labyrinthin, such as determined using an immunohistochemical (IHC) technique. In some embodiments, the individual is selected for treatment when a cancer sample from the individual shows that about 10% or greater, such as at least about any of 15%, 20%, 25%, 30%, 35%, 40%, 45%, 50%, 55%, 60%, 65%, 75%, 80%, 85%, 90%, or 95%, of a subpopulation of cancerous cells in the sample, such as cells at the periphery of a tumor, are positive for Labyrinthin, such as determined using an immunohistochemical (IHC) technique.

In some embodiments, the individual is human.

The dose of the anti-LAB construct compositions administered to an individual (such as a human) may vary with the particular composition, the mode of administration, and the type of disease being treated. In some embodiments, the amount of the composition is effective to result in an objective response (such as a partial response or a complete response). In some embodiments, the amount of an anti-LAB construct (e.g., full-length anti-LAB antibody, multi-specific anti-LAB molecule, or anti-LAB immunoconjugate) in the composition is included in a range of, e.g., about 0.001 pg to about 1000 pg. In some embodiments of any of the above aspects, the effective amount of an anti-LAB construct (e.g., full-length anti-LAB antibody, multi-specific anti-LAB molecule, or anti-LAB immunoconjugate) in the composition is in the range of about 0.1 pg/kg to about 100 mg/kg of total body weight.

The anti-LAB construct compositions can be administered to an individual (such as human) via various routes, including, for example, intravenous, intra-arterial, intraperitoneal, intrapulmonary, oral, inhalation, intravesicular, intramuscular, intra-tracheal, subcutaneous, intradermal, intraocular, intrathecal, transmucosal, and transdermal. In some embodiments, sustained continuous release formulation of the composition may be used.

The present application also provides methods of using an anti-LAB CAR or anti-LAB TCR to redirect the specificity of an effector cell (such as an immune cell, for example T cell) to a cell expressing Labyrinthin. Thus, the present application also provides a method of stimulating an effector cell-mediated response (such as a T cell-mediated immune response) to a target cell population or tissue expressing Labyrinthin in a mammal, comprising the step of administering to the mammal an effector cell (such as a T cell) that expresses an anti-LAB CAR and anti-LAB TCR.

Anti-LAB CAR or anti-LAB TCR effector cells (such as T cells) expressing the anti-LAB CAR can be infused to a recipient in need thereof. The infused cell is able to kill Labyrinthin-expressing cells in the recipient. In some embodiments, unlike antibody therapies, anti-LAB CAR or anti-LAB TCR effector cells (such as T cells) are able to replicate in vivo resulting in long-term persistence that can lead to sustained tumor control.

Ex vivo procedures are well known in the art and are discussed more fully below. Briefly, cells are isolated from a mammal (preferably a human) and genetically modified (i.e., transduced or transfected in vitro) with a vector expressing an anti-LAB CAR or anti-LAB TCR disclosed herein. The anti-LAB CART or anti-LAB TCRT cell can be administered to a mammalian recipient to provide a therapeutic benefit. The mammalian recipient may be a human and the anti-LAB CART or anti-LAB TCRT cell can be autologous with respect to the recipient. Alternatively, the cells can be allogeneic, syngeneic or xenogeneic with respect to the recipient.

In addition to using a cell-based vaccine in terms of ex vivo immunization, the present application also provides compositions and methods for in vivo immunization to elicit an immune response directed against an antigen in a patient.

The precise amount of the anti-LAB CAR or anti-LAB TCR effector cell (such as T cell) compositions of the present application to be administered can be determined by a physician with consideration of individual differences in age, weight, tumor size, extent of infection or metastasis, and condition of the patient (subject). In some embodiments, a pharmaceutical composition comprising the anti-LAB CAR or anti-LAB TCR effector cells (such as T cells) is administered at a dosage of about 10⁴ to about 10⁹ cells/kg body weight. Anti-LAB CAR or anti-LAB TCR effector cell (such as T cell) compositions may also be administered multiple times at these dosages. The cells can be administered by using infusion techniques that are commonly known in immunotherapy. The optimal dosage and treatment regimen for a particular patient can readily be determined by one skilled in the art of medicine by monitoring the patient for signs of disease and adjusting the treatment accordingly.

The administration of the anti-LAB CAR or anti-LAB TCR effector cells (such as T cells) may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In some embodiments, the anti-LAB CAR or anti-LAB TCR effector cell (such as T cell) compositions of the present application are administered to a patient by intradermal or subcutaneous injection. In some embodiments, the anti-LAB CAR or anti-LAB TCR effector cell (such as T cell) compositions of the present application are administered by i.v. injection. The compositions of anti-LAB CAR or anti-LAB TCR effector cells (such as T cells) may be injected directly into a tumor, lymph node, or site of infection.

The methods disclosed herein may comprise administering to the individual a plurality of doses of the anti-LAB constructs and/or compositions described herein over a period of time. In some embodiments, the dosing frequency and or dosage amount of an anti-LAB constructs and/or compositions described herein is adjusted over the course of the treatment, based on the judgment of the administering physician.

The methods for treatment and/or prevention disclosed herein are useful for treating or preventing a proliferative disease, such as a cancer, in an individual. In some embodiments, the cancer is a Labyrinthin-expressing cancer, such as a Labyrinthin-positive cancer. In some embodiments, the cancer is an adenocarcinoma. In some embodiments, the cancer is an early stage cancer, a non-metastatic cancer, a primary cancer, an advanced cancer, a locally advanced cancer, a metastatic cancer, a cancer in remission, a recurrent cancer, a resistant cancer, or a refractory cancer. In some embodiments, the cancer is a localized resectable cancer (e.g., a tumor that is confined to a portion of an organ that allows for complete surgical removal), a localized unresectable cancer (e.g., a localized tumor that is unresectable because crucial blood vessel structures), or an unresectable cancer. In some embodiments, the cancer is, according to TNM classifications, a stage I tumor, a stage II tumor, a stage III tumor, a stage IV tumor, a N1 tumor, or a M1 tumor.

The methods disclosed herein are useful for treating or preventing a cancer, such as a Labyrinthin-expressing cancer in an individual. In some embodiments, the individual has one or more of the following characteristics: (i) ability to understand and willingness to sign an informed consent form; (ii) at least 18 years of age with histologically confirmed adenocarcinoma and/or a Labyrinthin-expressing cancer; (iii) previously treated with at least 1 prior systemic therapy (chemotherapy and/or biologic therapy) and either had no response/progressed during treatment or progressed following the completion of systemic therapy or refuses all other treatment; (iv) tumor(s) must overexpress the Labyrinthin antigen, as determined by a screening immunohistochemical evaluation of the paraffin-embedded archival specimen demonstrating >10% of malignant cells staining for the antigen and with an intensity of at least 2× background according to the scoring by a single reference pathologist; (v) any number of prior chemotherapy regimens; (vi) documentation of delayed type hypersensitivity (DTH) response to common recall antigens prior to 1st vaccine injection; (vii) a performance status ≥60% on the Kamofsky scale; (viii) a life expectancy of ≥6 months at the time of treatment; (ix) measurable or evaluable disease; (x) a pretreatment absolute granulocyte count (AGC)≥1,000 and a pretreatment platelet count of ≥75,000 obtained within 4 weeks prior to 1^{st} vaccine injection; (xi) a pretreatment serum creatinine of ≤1.5 mg/dl is required; and (xii) a serum bilirubin≤1.5 and AST≤2.5× institutional upper limits of normal (≤5× if with liver metastases).

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXAMPLES

### Example 1

This example demonstrates the development and testing of a functional single-chain variable fragments (scFv) and an antigen-binding fragment (Fab) derived from an anti-Labyrinthin antibody (MCA 44-3A6).

Heavy chain variable domain (VH) and light chain variable domain (VH) cDNAs of MCA 44-3A6 were cloned with oligo primers useful for cDNA synthesis and PCR methods. Cloning was based on sequences published in the database of immunoglobulins. In brief, RNA prepared from about 5×10⁸ hybridoma cells was used for preparation of cDNA by commercially available reagents. Amplified cDNA of the VH and VL were digested with restriction enzymes, the sites of which sites were installed during PCR amplification. The genes were then subcloned into a phage display vector to construct a scFv for the purpose of library selection.

Libraries of scFv were then subcloned. For each scFV the sequence was fused so that upon subsequent expression the product would have a C-terminal myc tag. For phage library selection, protein antigen (histidine-tagged Labyrinthin) was immobilized onto a 96-well plate overnight. Aliquots of library phage were placed in assigned wells. Bound phages were eluted and added to TG1 culture for infection, and then rescued by KO7 helper phage. The infected TG1 cells were grown (30° C. overnight) and the bacterial culture supernatants harvested, precipitated, and resuspended in PBS for the next round of selection or phage ELISA.

For antigen preparation, the extracellular domain of Labyrinthin was cloned and expressed as a GST fusion protein using pGEX-2T. A crude preparation was readily prepared using size exclusion chromatography, and a pure preparation was made by affinity chromatography. It was observed that due to negatively charged and acidic residues in some portions of the protein that Labyrinthin strongly aggregates with progressive purification. Therefore, a histidine-tagged modified Labyrinthin protein (less the first 22 amino acids which are very hydrophobic: leader like sequence) was produced and used for the antigen-antibody screening procedure above (phage display). Though histidine is not expected to produce a major effect on Labyrinthin secondary structure, given the issue surrounding purification of this protein, an alternative KLH-conjugated peptide was also synthesized corresponding to amino acids #109-130 that contains the known binding site of MCA 44-3A6. Thus, although modified His-Labyrinthin was used in the page display antibody selection, the synthetic peptide was also included in the Biacore binding measurements to ensure optimal antibody candidate selection.

For Phage ELISA, His-Labyrinthin was coated onto ELISA plates (4° C. overnight). An aliquot of each phage to be tested was added to a respective well (incubated at 37° C. for 30-90 min) and then washed extensively with PBS. The phage bound-antigens were detected by incubation with HRP-conjugated anti-M13 antibody.

Subsequently, purified protein Labyrinthin antigens (His-Labyrinthin and synthetic peptide) were immobilized on a CM5 chip by amine coupling for Biacore measurements. Various concentrations of the purified antibody lead variants (selected from the phage libraries) were injected into the system to evaluate association and dissociation of the antibody-antigen interactions. The antibody affinity measurements were conducted on a Biacore 1000 instrument according to the manufacturer's protocol (Biacore, Inc., Piscataway, N.J.). Depending on how close the measured affinities were to each other for the different clones, the top three candidates for final screening and growth were stored and the highest affinity candidate was further evaluated using in vitro screening.

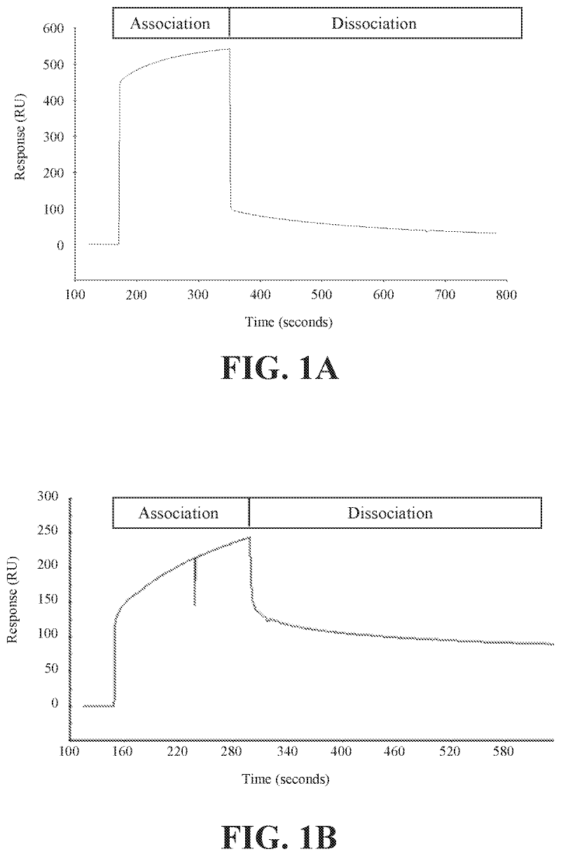

As shown in FIG. 1A and FIG. 1B, initial binding assays of the cloned and expressed protein (clone X373 scFv; FIG. 1B) and the parental MCA 44-3A6 antibody (FIG. 1A) using the His-tagged Labyrinthin were performed. X373 scFV was measured to have a dissociation constant (Kd) that was about four times lower than the parental MCA 44-3A6 antibody. Specifically, the Kd of X373 scFv was measured to be 4.8×10⁻⁷ M and the Kd of MCA 44-3A6 was measured to be 1.17×10⁻⁷ M.

Modifying the above-described development strategy, additional libraries were produced for an Fab format. A Fab, X509Fab, was identified through screening and was subsequently assessed for affinity to His-Labyrinthin and a KLH-conjugated Labyrinthin-derived peptide using the Biacore methods detailed above. Affinity measurements for X509Fab are reported in Table 5.

TABLE 5
Affinity measurements for X509Fab.

| | Kon/SE | Koff/SE | Kd (M) |
|---|---|---|---|
| His-Labyrinthin | 3.23E−04/826 | 2.58E−03/3.89E−05 | 8.0e−8 |
| protein | | | |
| KLH-conjugated | 5.48E+04/1260 | 1.28E−03/3.19E−05 | 2.33e−8 |
| Labyrinthin-derived | | | |
| peptide | | | |

X509Fab was subsequently tested for the ability to recognize native Labyrinthin in adenocarcinoma cells. Serial sections of A549 xenograft tumor sections (˜6 μm thickness) were used to compare X509Fab with the parental MCA 44-3A6 antibody within a near identical environment. The tissue sections exposed to 10 μg/ml of the antibodies (or no primary antibody) were subsequently developed with the appropriate secondary antibodies (HRP-conjugated goat anti-mouse or anti-human IgG) and DAB using the standard ABC method (Vector Laboratory, Burlingame, Calif.). The results (not shown) demonstrated that the X509Fab bound to native Labyrinthin and is at least equal to the parental antibody in the ability to recognize the epitope (Labyrinthin) on cancer cells.

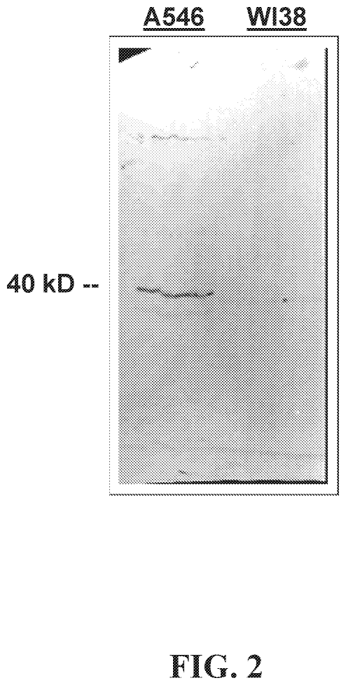

To determine whether the positive cell binding results of X509Fab represents specific recognition of Labyrinthin, standard western immunoblotting was performed with WI39 normal human fibroblasts (negative control) and A549 lung adenocarcinoma cell (positive control) lysates. A clear band was seen at the expected size for Labyrinthin (˜40 kD), whereas no signal was obtained in the negative control (FIG. 2). As with previous results using MCA 44-3A6, there was a faint lower band (breakdown product) and higher band (˜80 kD) confirming that Labyrinthin self-aggregates (FIG. 2).

A summary of affinity measurements for the scFv and Fab derived from an anti-Labyrinthin antibody (MCA 44-3A6) to His-Labyrinthin and a KLH-conjugated Labyrinthin-derived peptide are shown in Table 6.

TABLE 6
Affinity measurements of X373 scFv and X509Fab.

| | His- | KLH-conjugated Labyrinthin- |
|---|---|---|
| | Labyrinthin | derived peptide |
| MCA 44-3A6 | 117 nM | N/A |
| X373 scFv | 479 nM | 277 nM |
| X509Fab | 80 nM | 23 nM |

What is claimed is:

1. An isolated anti-Labyrinthin construct comprising an antibody moiety that specifically binds to Labyrinthin or a portion thereof and an effector domain.

2. The isolated anti-Labyrinthin construct of claim 1, wherein the antibody moiety is conjugated to the effector domain.

3. The isolated anti-Labyrinthin construct of claim 2, wherein the effector domain comprises an effector molecule.

4. The isolated anti-Labyrinthin construct of claim 3, wherein the effector molecule comprises a therapeutic agent.

5. The isolated anti-Labyrinthin construct of claim 4, wherein the therapeutic agent is selected from the group consisting of a drug, a toxin, a radioisotope, a protein, a peptide, and a nucleic acid.

6. The isolated anti-Labyrinthin construct of any one of claims 1-5, wherein the isolated anti-Labyrinthin construct is an antibody drug conjugate (ADC).

7. The isolated anti-Labyrinthin construct of claim 3, wherein the effector domain comprises a diagnostic agent.

8. The isolated anti-Labyrinthin construct of claim 7, wherein the diagnostic agent is a label.

9. The isolated anti-Labyrinthin construct of claim 1, wherein the antibody moiety is fused to an effector domain.

10. The isolated anti-Labyrinthin construct of claim 9, wherein the isolated anti-Labyrinthin construct is a chimeric antigen receptor (CAR) comprising an extracellular domain comprising the antibody moiety fused to the effector domain, wherein the effector domain comprises a transmembrane domain and an intracellular signaling domain.

11. The isolated anti-Labyrinthin construct of claim 9, wherein the isolated anti-Labyrinthin construct is a bispecific T-cell engager (BiTE) comprising an extracellular domain comprising the antibody moiety fused to an anti-CD3 antibody or fragment thereof.

12. The isolated anti-Labyrinthin construct of any one of claims 1-9, wherein the isolated anti-Labyrinthin construct is a T-cell receptor (TCR) comprising an extracellular domain comprising the antibody moiety fused to the effector domain, wherein the effector domain comprises a transmembrane domain and/or an intracellular signaling domain of a TCR subunit.

13. The isolated anti-Labyrinthin construct of any one of claims 1-12, wherein the antibody moiety is a full-length antibody, a Fab, a Fab′, a (Fab′)2, an Fv, or a single chain Fv (scFv).

14. The isolated anti-Labyrinthin construct of any one of claims 1-13, wherein the antibody moiety binds Labyrinthin or a portion thereof with a Kd from about 0.1 pM to about 500 nM.

15. The isolated anti-Labyrinthin construct of any one of claims 1-14, wherein the antibody moiety specifically binds to a Labyrinthin-derived peptide comprising an amino acid sequence selected from the group consisting of: SEQ ID NOs:2-32.

16. The isolated anti-Labyrinthin construct of claim 15, wherein the Labyrinthin-derived peptide comprises a B-cell epitope.

17. The isolated anti-Labyrinthin construct of claim 15 or 16, wherein the Labyrinthin-derived peptide comprises a T-cell epitope.

18. The isolated anti-Labyrinthin construct of any one of claims 1-17, wherein the antibody moiety is multispecific.

19. The isolated anti-Labyrinthin construct of claim 18, wherein the multispecific antibody moiety is a tandem scFv, a diabody (db), a single chain diabody (scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody.

20. The isolated anti-Labyrinthin construct of claim 19, wherein the multispecific antibody moiety is a tandem scFv comprising two scFvs linked by an optional peptide linker.

21. A pharmaceutical composition comprising the isolated anti-Labyrinthin construct of any one of claims 1-20.

22. A host cell expressing the isolated anti-Labyrinthin construct of any one of claims 1-20.

23. A nucleic acid encoding the polypeptide components of the isolated anti-Labyrinthin construct of any one of claims 1-20.

24. An effector cell comprising the nucleic acid of claim 23.

25. The effector cell of claim 24, wherein the effector cell is a T cell.

26. A method of detecting a cell presenting Labyrinthin or a portion thereof on its surface, comprising:
(a) contacting the cell with the isolated anti-Labyrinthin construct of claim 8; and
(b) detecting the presence of the label on the cell.

27. A method of treating an individual having a Labyrinthin-positive disease, comprising administering to the individual:
(a) an effective amount of the pharmaceutical composition of claim 21; or
(b) an effective amount of the effector cell of claim 24 or 25.

28. The method of claim 27, wherein the Labyrinthin status is used as a basis for selecting the individual for treatment.

29. A method of diagnosing an individual having a Labyrinthin-positive disease, comprising:
(a) administering an effective amount of the isolated anti-Labyrinthin construct of claim 8 to the individual; and
(b) determining the level of the label in the individual, wherein a level of the label above a threshold level indicates that the individual has the Labyrinthin-positive disease.

30. A method of diagnosing an individual having a Labyrinthin-positive disease, comprising:
(a) contacting a sample derived from the individual with the isolated anti-Labyrinthin construct of any one of claims 1-20; and
(b) identifying one or more cells bound with the isolated anti-Labyrinthin construct in the sample,
thereby diagnosing an individual having a Labyrinthin-positive disease.

31. The method of any one of claims 25-29, wherein the Labyrinthin-positive disease is Labyrinthin-positive cancer.

32. The method of claim 31, wherein the Labyrinthin-positive cancer is an adenocarcinoma.
